(19) United States
(12) Patent Application Publication
(10) Pub. No.: US 2022/0288278 A1
(43) Pub. Date: Sep. 15, 2022

(54) ULTRASOUND MEDIATED POLYMERIZATION FOR CELL DELIVERY, DRUG DELIVERY AND 3D PRINTING

(71) Applicant: Technion Research & Development Foundation Limited, Haifa (IL)

(72) Inventors: Shulamit LEVENBERG, Haifa (IL); Lior DEBBI, Haifa (IL); Majd MACHOUR, Haifa (IL); Haim AZHARI, Haifa (IL); Daniel DAHIS, Haifa (IL)

(73) Assignee: Technion Research & Development Foundation Limited, Haifa (IL)

(21) Appl. No.: 17/685,512

(22) Filed: Mar. 3, 2022

Related U.S. Application Data
(60) Provisional application No. 63/156,968, filed on Mar. 5, 2021.

(51) Int. Cl. A61L 27/54; A61L 27/50; B33Y 80/00; B33Y 40/20; B33Y 50/02; A61L 27/26; B33Y 10/00; B33Y 70/00

(52) U.S. Cl. CPC A61L 27/54; A61L 27/50; B33Y 80/00; B33Y 40/20; B33Y 50/02; A61L 27/26; B33Y 10/00; B33Y 70/00; B33Y 30/00

(57) ABSTRACT

An aspect of the invention relates to methods and implants comprising acoustic-sensitive material and at least one additional component within said acoustic-sensitive material. In some embodiments, the at least one additional component is one or more of at least one releasable drug within said acoustic-sensitive material and/or a plurality of cells within said acoustic-sensitive material. In some embodiments, the implant comprises a dedicated form, which is provided inside the body of the patient.

### RELATED APPLICATIONS

This application claims the benefit of priority of U.S. Provisional Patent Application No. 63/156,968 filed on Mar. 5, 2021, the contents of which are incorporated herein by reference in their entirety.

### FIELD AND BACKGROUND OF THE INVENTION

The present invention, in some embodiments thereof, relates to polymerization of implants and, more particularly, but not exclusively, to a polymerization of implants inside the body with optionally performing an additional action.

The field of 3D bioprinting has developed rapidly in recent years with the aim to treat various types of pathologies by fabricating therapeutics synthetic or biological implants and engineered tissues for restoring tissue or organ level function. Recently the idea of in-situ bioprinting (i.e. printing directly in the patient's body) has been raised with the inherent advantages of direct material delivery into the natural environment which will accelerate scaffold integration and tissue restoration with real-time patient specific spatial fitting. However, one major hurdle of this novel approach is the limited ability to access deep tissues without performing highly invasive procedures.

Additional background art includes U.S. Patent Application Publication No. US2002165582A1 disclosing an apparatus, method and composition for embolization of a vascular site in a blood vessel. The composition is introduced via catheter to the vascular site and activated by an activator introduced by the catheter or external means. The composition polymerizes or precipitates in situ via the activation provided by the catheter or external means.

U.S. Patent Application Publication No. US2003194505A1 disclosing a process for accelerating the polymerization of an implant. Specifically, a process for accelerating the bond between a surgical adhesive and tissue. The accelerated bonding is achieved by applying radio and/or acoustic energy to the adhesive/tissue interface such that the adhesive is coupled to the energy and absorbs a substantial quantity of the applied energy. The process comprising the steps of: a) applying said adhesive to tissue or bone, b) applying radio and/or acoustic energy to the adhesive deposited on the tissue or bone, c) dissipating the applied energy within the adhesive so as to promote adhesive/fluid mixing at the adhesive/tissue interface, d) dissipating the applied energy within the adhesive so as to activate chemical bonding at the adhesive/tissue interface, and e) dissipating the applied energy within the adhesive so as to increase the reaction rate both of the internal polymerization of the adhesive and of the adhesive/tissue interface.

U.S. Pat. No. 8,241,324B2 disclosing the use of a low frequency ultrasonic device for the delivery and activation of collagen based foam sealants to a human and/or animal patient for sealing puncture wounds in vascular tissues. The ultrasonic vascular closure device comprises an ultrasonic generator, an ultrasound transducer, a chamber containing a foam sealant, a transducer tip, a radiation surface, an orifice located at the distal end of the chamber. The foam sealant is ejected into a puncture wound and activated with ultrasonic waves emitting from the radiation surface. The ultrasonic waves induce vibrations within the foam sealant, slightly warming the foam sealants to assist the rapid sealing the puncture. The ultrasonic waves also provide and anesthetic effect for the pain and discomfort from the puncture site.

U.S. Patent Application Publication No. US2004030341A1 disclosing implants that form positive connections with human or animal tissue parts, particularly bones, implants consisting of a material that can be liquefied by means of mechanical energy. Particularly suitable materials of this type are thermoplastics (e.g. resorbable thermoplastics) or thixotropic materials. The implants are brought into contact with the tissue part, are subjected to the action of ultrasonic energy and are simultaneously pressed against the tissue part. The liquefiable material then liquefies and is pressed into openings or surface asperities of the tissue part so that, once solidified, it is positively joined thereto. The implantation involves the use of an implantation device comprising a generator, an oscillating element and a resonator, whereby the generator causes the oscillating element to mechanically oscillate, and the element transmits the oscillations to the resonator.

U.S. Patent Application Publication No. US2017342220A1 disclosing a polymer gel and methods of preparing thereof having mechanical strength and an ability to maintain surface wetness for a longer time.

U.S. Patent Application Publication No. US2019239868A1 disclosing an implant for use in a body, at least one portion of the surface of the implant being mutually engageable with at least one portion of at least one body part. Also disclosed is a method of surgery comprising the steps of: forming an implant comprising at least one portion of the surface of the implant being mutually engageable with at least one portion of at least one body part, applying a layer of adhesive to the at least one portion of the surface, and engaging the at least one portion of the surface with the at least one portion of at least one body part.

U.S. Patent Application Publication No. US2008132899A1 disclosing implantable bone fill materials, systems and methods of treating bone abnormalities such as compression fractures of vertebrae, bone necrosis, bone tumors, cysts and the like. In an exemplary embodiment, the bone abnormality is accessed and a space is created by bone removal or compaction. An exemplary implant of the invention has a substantially fluid impermeable surface portion and an interior portion including an in-situ hardenable bone cement. The method of the invention includes applying energy to the fill material to accelerate polymerization and hardening of the material for supporting the bone.

An online article (https://physicsworld(dot)com/a/tissue-engineering-moves-closer-to-3d-printing-inside-the-body/) discloses a specially-formulated bioink designed for printing directly in the body. They used the hydrogel gelatin methacryloyl (GelMA) as the biomaterial, and introduced Laponite and methylcellulose as rheological modifiers to enhance printability. It is disclosed also that the researchers used the GelMA/Laponite/methylcellulose (GLM) formulation, with and without encapsulated fibroblasts, to construct complex 3D tissue scaffolds with clinically relevant dimensions and consistent structures. They claimed to have successfully 3D printed the scaffolds on agarose and chicken breast pieces, using on-site crosslinking with visible light. For cell-laden GLM, the fibroblasts exhibited consistent mechanical properties and a viability of 71-77% over 21 days in the printed scaffolds. A scientific article on this topic was also published (Direct-write 3D printing and characterization of a GelMA-based biomaterial for intracorporeal tissue engineering, A Asghari Adib et al 2020 Biofabrication 12 045006), and an U.S. Patent Application Publication No. 20200324469A1 disclosing systems and methods for in vivo multi-material bioprinting. The in vivo multi-material bioprinting can be used to fabricate biomedical constructs within a patient minimally invasively. The systems and methods can utilize a multi-material bioprinter, which includes a biocompatible portion. The biocompatible portion can include a single printhead for in vivo bioprinting. The single printhead can include a plurality of outlets, each linked to one of a plurality of reservoirs. Each of the plurality of reservoirs can each house a different bioink for bioprinting. Each of the plurality of outlets can be activated to release a respective bioink.

A scientific article by Tamay Dilara Goksu et al. discloses that Three-dimensional (3D) and Four-dimensional (4D) printing have emerged as the next generation of fabrication techniques, spanning across various research areas, such as engineering, chemistry, biology, computer science, and materials science. Three-dimensional printing enables the fabrication of complex forms with high precision, through a layer-by-layer addition of different materials. Use of intelligent materials which change shape or color, produce an electrical current, become bioactive, or perform an intended function in response to an external stimulus, paves the way for the production of dynamic 3D structures, which is now called 4D printing. 3D and 4D printing techniques have great potential in the production of scaffolds to be applied in tissue engineering, especially in constructing patient specific scaffolds. Furthermore, physical and chemical guidance cues can be printed with these methods to improve the extent and rate of targeted tissue regeneration. It is disclosed a survey of 3D and 4D printing methods, and the advantage of their use in tissue regeneration over other scaffold production approaches (https://www(dot)frontiersin(dot)org/article/10(dot)3389/fbioe(dot)2019(dot)00164).

Australian Patent Application Publication No. 2018201765A1 discloses that to address the limitations of existing 3D printing processes restricted to two dimensional layer by layer and surface printing methods, it is proposed to provide a 3D printing process using high intensity focused ultrasound (HIFU) technology to produce deep three dimensional formations within a medium using a three dimensional toolpath motion. Ultrasound waves penetrate plurality of mediums with matching acoustic impedance with the HIFU transducer concentrating energy into a focal point to stimulate temperate increase at desired location. Thermoresponsive material is contained within the liquid medium whereby a phase transition occurs from liquid to gel or solid upon heating above its transition temperature. The proposed process is able to be utilized in bioprinting to produce tissue and organ cell formations using biocompatible materials and whereby the formations are able to be produced in-vivo within the patient.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. An implant, comprising:

a. acoustic-sensitive material, and

b. at least one releasable drug within said acoustic-sensitive material.

Example 2. The implant according to example 1, wherein said acoustic-sensitive material comprises materials with functional acrylate or diacrylate or methacrylate groups.
Example 3. The implant according to example 1 or example 2, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 4. The implant according to any one of examples 1-3, wherein said acoustic-sensitive material further comprises a plurality of cells within said acoustic-sensitive material.
Example 5. The implant according to any one of examples 1-4, wherein said acoustic-sensitive material hardens when exposed to ultrasound emissions.
Example 6. The implant according to any one of examples 1-5, wherein said ultrasound emissions are characterized by low frequencies.
Example 7. The implant according to any one of examples 1-6, wherein said ultrasound emissions are from about 30 kHz to about 1000 kHz.
Example 8. The implant according to any one of examples 1-7, wherein said ultrasound emissions are emitted for a period of time of from about 5 seconds to about 30 seconds.
Example 9. The implant according to any one of examples 1-8, wherein said ultrasound emissions are emitted for a period of time of from about 3 seconds to about 120 seconds.
Example 10. The implant according to any one of examples 1-9, wherein said ultrasound emissions are emitted for a period of time of more than 2 seconds.
Example 11. The implant according to any one of examples 1-10, wherein said ultrasound emissions are characterized by an intensity range of from about 0.5 Watt/cm2 to about 2.2 Watt/cm2.
Example 12. The implant according to any one of examples 1-11, wherein said ultrasound emissions are characterized by an intensity range of from about 0.1 Watt/cm2 to about 10 Watt/cm2.
Example 13. The implant according to any one of examples 1-12, wherein said implant is printed within a supportive subtract.
Example 14. The implant according to any one of examples 1-13, wherein said printed within said supportive material is performed before implantation of said implant.
Example 15. The implant according to any one of examples 1-14, wherein said printed within said supportive material is performed after implantation of said implant.
Example 16. The implant according to any one of examples 1-15, wherein said supportive material comprises one or more of agar, gelatin and Pluronic F-127.
Example 17. The implant according to any one of examples 1-16, wherein said supportive material is washable away.
Example 18. The implant according to any one of examples 1-17, wherein said implant comprises a dedicated form when focused ultrasound is applied to said implant according to a predetermined CAD model layer.
Example 19. The implant according to any one of examples 1-18, wherein said ultrasound emissions are delivered via planar ultrasound transducers.
Example 20. The implant according to any one of examples 1-19, wherein said acoustic-sensitive material comprises a solution of pre-polymer and acoustic-sensitive cross-linker loaded micro-capsules.
Example 21. The implant according to any one of examples 1-20, wherein said acoustic-sensitive cross-linker loaded micro-capsules comprise liposomes including said cross-linker.
Example 22. The implant according to any one of examples 1-21, wherein said pre-polymer comprises alginate.
Example 23. An implant, comprising:

a. acoustic-sensitive material, and

b. a plurality of cells within said acoustic-sensitive material.

Example 24. The implant according to example 23, wherein said acoustic-sensitive material comprises materials with functional acrylate or diacrylate or methacrylate groups.
Example 25. The implant according to example 23 or example 24, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 26. The implant according to any one of examples 23-25, wherein said acoustic-sensitive material further comprises at least one releasable drug within said acoustic-sensitive material.
Example 27. The implant according to any one of examples 23-26, wherein said acoustic-sensitive material hardens when exposed to ultrasound emissions.
Example 28. The implant according to any one of examples 23-27, wherein said ultrasound emissions are characterized by low frequencies.
Example 29. The implant according to any one of examples 23-28, wherein said ultrasound emissions are from about 30 kHz to about 1000 kHz.
Example 30. The implant according to any one of examples 23-29, wherein said ultrasound emissions are emitted for a period of time of from about 5 seconds to about 30 seconds.
Example 31. The implant according to any one of examples 23-30, wherein said ultrasound emissions are emitted for a period of time of from about 3 seconds to about 120 seconds.
Example 32. The implant according to any one of examples 23-31, wherein said ultrasound emissions are emitted for a period of time of more than 2 seconds.
Example 33. The implant according to any one of examples 23-32, wherein said ultrasound emissions are characterized by an intensity range of from 0.5 Watt/cm2 to about 2.2 Watt/cm2.
Example 34. The implant according to any one of examples 23-33, wherein said ultrasound emissions are characterized by an intensity range of from 0.1 Watt/cm2 to about 10 Watt/cm2.
Example 35. The implant according to any one of examples 23-34, wherein said implant is printed within a supportive subtract.
Example 36. The implant according to any one of examples 23-35, wherein said printed within said supportive material is performed before implantation of said implant.
Example 37. The implant according to any one of examples 23-36, wherein said printed within said supportive material is performed after implantation of said implant.
Example 38. The implant according to any one of examples 23-37, wherein said supportive material comprises one or more of agar, gelatin and Pluronic F-127.
Example 39. The implant according to any one of examples 23-38, wherein said supportive material is washable away.
Example 40. The implant according to any one of examples 23-39, wherein said implant comprises a dedicated form when focused ultrasound is applied to said implant according to a predetermined CAD model layer.
Example 41. The implant according to any one of examples 23-40, wherein said ultrasound emissions are delivered via planar ultrasound transducers.
Example 42. The implant according to any one of examples 23-41, wherein said acoustic-sensitive material comprises a solution of pre-polymer and acoustic-sensitive cross-linker loaded micro-capsules.
Example 43. The implant according to any one of examples 23-42, wherein said acoustic-sensitive cross-linker loaded micro-capsules comprise liposomes including said cross-linker.
Example 44. The implant according to any one of examples 23-43, wherein said pre-polymer comprises alginate.
Example 45. An implant system, comprising:

a. an ultrasound transducer; and

b. an implant comprising:
i. acoustic-sensitive material, and
ii. at least one component within said acoustic-sensitive material.
   Example 46. The system according to example 45 wherein said acoustic-sensitive material comprises materials with functional acrylate or diacrylate or methacrylate groups.
   Example 47. The system according to example 45 or example 46, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
   Example 48. The system according to any one of examples 45-47, wherein said acoustic-sensitive material further comprises a plurality of cells within said acoustic-sensitive material.
   Example 49. The system according to any one of examples 45-48, wherein said acoustic-sensitive material further comprises at least one releasable drug within said acoustic-sensitive material.
   Example 50. The system according to any one of examples 45-49, wherein said acoustic-sensitive material hardens when exposed to ultrasound emissions provided by said ultrasound transducer.
   Example 51. The system according to any one of examples 45-50, wherein said ultrasound emissions are characterized by low frequencies.
   Example 52. The system according to any one of examples 45-51, wherein said ultrasound emissions are from about 30 kHz to about 1000 kHz.
   Example 53. The system according to any one of examples 45-52, wherein said ultrasound emissions are emitted for a period of time of from about 5 seconds to about 30 seconds.
   Example 54. The system according to any one of examples 45-53, wherein said ultrasound emissions are emitted for a period of time of from about 3 seconds to about 120 seconds.
   Example 55. The system according to any one of examples 45-54, wherein said ultrasound emissions are emitted for a period of time of more than 2 seconds.
   Example 56. The system according to any one of examples 45-55, wherein said ultrasound emissions are characterized by an intensity range of from about 0.5 Watt/cm2 to about 2.2 Watt/cm2.
   Example 57. The system according to any one of examples 45-56, wherein said ultrasound emissions are characterized by an intensity range of from about 0.1 Watt/cm2 to about 10 Watt/cm2.
   Example 58. The system according to any one of examples 45-57, wherein said implant is printed within a supportive subtract.
   Example 59. The system according to any one of examples 45-58, wherein said printed within said supportive material is performed before implantation of said implant.
   Example 60. The system according to any one of examples 45-59, wherein said printed within said supportive material is performed after implantation of said implant.
   Example 61. The system according to any one of examples 45-60, wherein said supportive material comprises one or more of agar, gelatin and Pluronic F-127.
   Example 62. The system according to any one of examples 45-61, wherein said supportive material is washable away.
   Example 63. The system according to any one of examples 45-62, wherein said implant comprises a dedicated form when focused ultrasound is applied to said implant according to a predetermined CAD model layer.
   Example 64. The system according to any one of examples 45-63, wherein said ultrasound transducers are planar ultrasound transducers.
   Example 65. The system according to any one of examples 45-64, wherein said ultrasound transducers are focused ultrasound transducers.
   Example 66. The system according to any one of examples 45-65, wherein said acoustic-sensitive material comprises a solution of pre-polymer and acoustic-sensitive cross-linker loaded micro-capsules.
   Example 67. The system according to any one of examples 45-66, wherein said acoustic-sensitive cross-linker loaded micro-capsules comprise liposomes including said cross-linker.
   Example 68. The system according to any one of examples 45-67, wherein said pre-polymer comprises alginate.
   Example 69. A method of implanting an implant, comprising:

a. implanting acoustic-sensitive material in a first site of said patient;

b. selectively hardening said acoustic-sensitive material by emitting acoustic energy to a second site of said patient.

Example 70. The method according to example 69, wherein said first site and said second site are the same site.
Example 71. The method according to example 69 or example 70, wherein said first site and said second site are different sites.
Example 72. The method according to any one of examples 69-71, wherein said first site is an implantation target site.
Example 73. The method according to any one of examples 69-72, wherein said first site is a blood vessel.
Example 74. The method according to any one of examples 69-73, wherein said second site is said implantation target site.
Example 75. The method according to any one of examples 69-74, wherein said acoustic-sensitive material comprises materials with functional acrylate or diacrylate or methacrylate groups.
Example 76. The method according to any one of examples 69-75, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 77. The method according to any one of examples 69-76, wherein said acoustic-sensitive material further comprises a plurality of cells within said acoustic-sensitive material.
Example 78. The method according to any one of examples 69-77, wherein said acoustic-sensitive material further comprises at least one releasable drug within said acoustic-sensitive material.
Example 79. The method according to any one of examples 69-78, wherein said emitting acoustic energy comprises emitting ultrasound emissions.
Example 80. The method according to any one of examples 69-79, wherein said emitting comprises emitting at low frequencies.
Example 81. The method according to any one of examples 69-80, wherein said emitting comprises emitting at a frequency of from about 30 kHz to about 1000 kHz.
Example 82. The method according to any one of examples 69-81, wherein said emitting comprises emitting for a period of time of from about 5 seconds to about 30 seconds.
Example 83. The method according to any one of examples 69-82, wherein said emitting comprises emitting for a period of time of from about 3 seconds to about 120 seconds.
Example 84. The method according to any one of examples 69-83, wherein said emitting comprises emitting for a period of time of more than 2 seconds.
Example 85. The method according to any one of examples 69-84, wherein said ultrasound emissions are characterized by an intensity range of from about 0.5 Watt/cm2 to about 2.2 Watt/cm2.
Example 86. The method according to any one of examples 69-85, wherein said ultrasound emissions are characterized by an intensity range of from about 0.1 Watt/cm2 to about 10 Watt/cm2.
Example 87. The method according to any one of examples 69-86, wherein said selectively hardening is performed within a supportive subtract.
Example 88. The method according to any one of examples 69-87, wherein said selectively hardening within said supportive material is performed before implantation of said implant.
Example 89. The method according to any one of examples 69-88, wherein said selectively hardening within said supportive material is performed after implantation of said implant.
Example 90. The method according to any one of examples 69-89, wherein said supportive material comprises one or more of agar, gelatin and Pluronic F-127.
Example 91. The method according to any one of examples 69-90, wherein said method further comprises washing away said supportive material.
Example 92. The method according to any one of examples 69-91, wherein said method further comprises providing a dedicated form to said implant by emitting focused ultrasound to said implant according to a predetermined CAD model layer.
Example 93. The method according to any one of examples 69-92, wherein said emitting acoustic energy comprise emitting via planar ultrasound transducers.
Example 94. The method according to any one of examples 69-93, wherein said emitting acoustic energy comprise emitting via focused ultrasound transducers.
Example 95. The method according to any one of examples 69-94, wherein said acoustic-sensitive material comprises a solution of pre-polymer and acoustic-sensitive cross-linker loaded micro-capsules.
Example 96. The method according to any one of examples 69-95, wherein said acoustic-sensitive cross-linker loaded micro-capsules comprise liposomes including said cross-linker.
Example 97. The method according to any one of examples 69-96, wherein said pre-polymer comprises alginate.
Example 98. A method of providing a determined form to an implant inside a body of a patient, comprising:

a. preparing a virtual model of said implant;

b. preparing acoustic-sensitive material;

c. injecting said acoustic-sensitive material at a first location in said body of said patient;

d. selectively hardening said acoustic-sensitive material according to said virtual model of said implant at a second location in said body of said patient.

Example 99. The method according to example 98, wherein said first site and said second site are the same site.
Example 100. The method according to example 98 or example 99, wherein said first site and said second site are different sites.
Example 101. The method according to any one of examples 98-100, wherein said first site is an implantation target site.
Example 102. The method according to any one of examples 98-101, wherein said first site is a blood vessel.
Example 103. The method according to any one of examples 98-102, wherein said second site is said implantation target site.
Example 104. The method according to any one of examples 98-103, wherein said acoustic-sensitive material comprises materials with functional acrylate or diacrylate or methacrylate groups.
Example 105. The method according to any one of examples 98-104, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 106. The method according to any one of examples 98-105, wherein said acoustic-sensitive material further comprises a plurality of cells within said acoustic-sensitive material.
Example 107. The method according to any one of examples 98-106, wherein said acoustic-sensitive material further comprises at least one releasable drug within said acoustic-sensitive material.
Example 108. The method according to any one of examples 98-107, wherein said emitting acoustic energy comprises emitting ultrasound emissions.
Example 109. The method according to any one of examples 98-108, wherein said emitting comprises emitting at low frequencies.
Example 110. The method according to any one of examples 98-109, wherein said emitting comprises emitting at a frequency of from about 30 kHz to about 1000 kHz.
Example 111. The method according to any one of examples 98-110, wherein said emitting comprises emitting for a period of time of from about 5 seconds to about 30 seconds.
Example 112. The method according to any one of examples 98-111, wherein said emitting comprises emitting for a period of time of from about 3 seconds to about 120 seconds.
Example 113. The method according to any one of examples 98-112, wherein said emitting comprises emitting for a period of time of more than 2 seconds.
Example 114. The method according to any one of examples 98-113, wherein said ultrasound emissions are characterized by an intensity range of from about 0.5 Watt/cm2 to about 2.2 Watt/cm2.
Example 115. The method according to any one of examples 98-114, wherein said ultrasound emissions are characterized by an intensity range of from about 0.1 Watt/cm2 to about 10 Watt/cm2.
Example 116. The method according to any one of examples 98-115, wherein said selectively hardening is performed within a supportive subtract.
Example 117. The method according to any one of examples 98-116, wherein said selectively hardening within said supportive material is performed before implantation of said implant.
Example 118. The method according to any one of examples 98-117, wherein said selectively hardening within said supportive material is performed after implantation of said implant.
Example 119. The method according to any one of examples 98-118, wherein said supportive material comprises one or more of agar, gelatin and Pluronic F-127.
Example 120. The method according to any one of examples 98-119, wherein said method further comprises washing away said supportive material.
Example 121. The method according to any one of examples 98-120, wherein said method further comprises providing a dedicated form to said implant by emitting focused ultrasound to said implant according to a predetermined CAD model layer.
Example 122. The method according to any one of examples 98-121, wherein said emitting acoustic energy comprise emitting via planar ultrasound transducers.
Example 123. The method according to any one of examples 98-122, wherein said emitting acoustic energy comprise emitting via focused ultrasound transducers.
Example 124. The method according to any one of examples 98-123, wherein said acoustic-sensitive material comprises a solution of pre-polymer and acoustic-sensitive cross-linker loaded micro-capsules.
Example 125. The method according to any one of examples 98-124, wherein said acoustic-sensitive cross-linker loaded micro-capsules comprise liposomes including said cross-linker.
Example 126. The method according to any one of examples 98-125, wherein said pre-polymer comprises alginate.
Example 127. An implant, comprising:

a. acoustic-sensitive material comprising a solution of pre-polymer and an acoustic-sensitive cross-linker loaded micro-capsules; and

b. at least one releasable drug within said solution.

Example 128. The implant according to example 127, wherein said acoustic-sensitive cross-linker loaded micro-capsules comprise liposomes including said cross-linker.
Example 129. The implant according to example 127 or example 128, wherein said pre-polymer comprises alginate.
Example 130. The implant according to any one of examples 127-129, wherein said acoustic-sensitive material comprises materials with functional acrylate or diacrylate or methacrylate groups.
Example 131. The implant according to any one of examples 127-130, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 132. The implant according to any one of examples 127-131, wherein said acoustic-sensitive material further comprises a plurality of cells within said acoustic-sensitive material.
Example 133. The implant according to any one of examples 127-132, wherein said acoustic-sensitive material hardens when exposed to ultrasound emissions.
Example 134. The implant according to any one of examples 127-133, wherein said ultrasound emissions are characterized by low frequencies.
Example 135. The implant according to any one of examples 127-134, wherein said ultrasound emissions are from about 30 kHz to about 1000 kHz.
Example 136. The implant according to any one of examples 127-135, wherein said ultrasound emissions are emitted for a period of time of from about 5 seconds to about 30 seconds.
Example 137. The implant according to any one of examples 127-136, wherein said ultrasound emissions are emitted for a period of time of from about 3 seconds to about 120 seconds.
Example 138. The implant according to any one of examples 127-137, wherein said ultrasound emissions are emitted for a period of time of more than 2 seconds.
Example 139. The implant according to any one of examples 127-138, wherein said ultrasound emissions are characterized by an intensity range of from about 0.5 Watt/cm2 to about 2.2 Watt/cm2.
Example 140. The implant according to any one of examples 127-139, wherein said ultrasound emissions are characterized by an intensity range of from about 0.1 Watt/cm2 to about 10 Watt/cm2.
Example 141. The implant according to any one of examples 127-140, wherein said implant is printed within a supportive subtract.
Example 142. The implant according to any one of examples 127-141, wherein said printed within said supportive material is performed before implantation of said implant.
Example 143. The implant according to any one of examples 127-142, wherein said printed within said supportive material is performed after implantation of said implant.
Example 144. The implant according to any one of examples 127-143, wherein said supportive material comprises one or more of agar, gelatin and Pluronic F-127.
Example 145. The implant according to any one of examples 127-144, wherein said supportive material is washable away.
Example 146. The implant according to any one of examples 127-145, wherein said implant comprises a dedicated form when focused ultrasound is applied to said implant according to a predetermined CAD model layer.
Example 147. The implant according to any one of examples 127-146, wherein said ultrasound emissions are delivered via planar ultrasound transducers.
Example 148. An implant, comprising:

a. acoustic-sensitive material comprising a solution of pre-polymer and acoustic-sensitive cross-linker loaded micro-capsules; and

b. a plurality of cells within said acoustic-sensitive material.

Example 149. The implant according to example 148, wherein said acoustic-sensitive cross-linker loaded micro-capsules comprise liposomes including said cross-linker.
Example 150. The implant according to example 148 or example 149, wherein said pre-polymer comprises alginate.
Example 151. The implant according to any one of examples 148-150, wherein said acoustic-sensitive material comprises materials with functional acrylate or diacrylate or methacrylate groups.
Example 152. The implant according to any one of examples 148-151, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 153. The implant according to any one of examples 148-152, wherein said acoustic-sensitive material further comprises at least one releasable drug within said acoustic-sensitive material.
Example 154. The implant according to any one of examples 148-153, wherein said acoustic-sensitive material hardens when exposed to ultrasound emissions.
Example 155. The implant according to any one of examples 148-154, wherein said ultrasound emissions are characterized by low frequencies.
Example 156. The implant according to any one of examples 148-155, wherein said ultrasound emissions are from about 30 kHz to about 1000 kHz.
Example 157. The implant according to any one of examples 148-156, wherein said ultrasound emissions are emitted for a period of time of from about 5 seconds to about 30 seconds.
Example 158. The implant according to any one of examples 148-157, wherein said ultrasound emissions are emitted for a period of time of from about 3 seconds to about 120 seconds.
Example 159. The implant according to any one of examples 148-158, wherein said ultrasound emissions are emitted for a period of time of more than 2 seconds.
Example 160. The implant according to any one of examples 148-159, wherein said ultrasound emissions are characterized by an intensity range of from about 0.5 Watt/cm2 to about 2.2 Watt/cm2.
Example 161. The implant according to any one of examples 148-160, wherein said ultrasound emissions are characterized by an intensity range of from about 0.1 Watt/cm2 to about 10 Watt/cm2.
Example 162. The implant according to any one of examples 148-161, wherein said implant is printed within a supportive subtract.
Example 163. The implant according to any one of examples 148-162, wherein said printed within said supportive material is performed before implantation of said implant.
Example 164. The implant according to any one of examples 148-163, wherein said printed within said supportive material is performed after implantation of said implant.
Example 165. The implant according to any one of examples 148-164, wherein said supportive material comprises one or more of agar, gelatin and Pluronic F-127.
Example 166. The implant according to any one of examples 148-165, wherein said supportive material is washable away.
Example 167. The implant according to any one of examples 148-166, wherein said implant comprises a dedicated form when focused ultrasound is applied to said implant according to a predetermined CAD model layer.
Example 168. The implant according to any one of examples 148-167, wherein said ultrasound emissions are delivered via planar ultrasound transducers.
Example 169. An implant system, comprising:

a. an ultrasound transducer; and

b. an implant comprising:
i. acoustic-sensitive material comprising a solution of pre-polymer and acoustic-sensitive cross-linker loaded micro-capsules; and
ii. at least one component within said acoustic-sensitive material.
   Example 170. The system according to example 169, wherein said acoustic-sensitive cross-linker loaded micro-capsules comprise liposomes including said cross-linker.
   Example 171. The system according to example 169 or example 170, wherein said pre-polymer comprises alginate.
   Example 172. The system according to any one of examples 169-171, wherein said acoustic-sensitive material comprises materials with functional acrylate or diacrylate or methacrylate groups.
   Example 173. The system according to any one of examples 169-172, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
   Example 174. The system according to any one of examples 169-173, wherein said acoustic-sensitive material further comprises a plurality of cells within said acoustic-sensitive material.
   Example 175. The system according to any one of examples 169-174, wherein said acoustic-sensitive material further comprises at least one releasable drug within said acoustic-sensitive material.
   Example 176. The system according to any one of examples 169-175, wherein said acoustic-sensitive material hardens when exposed to ultrasound emissions provided by said ultrasound transducer.
   Example 177. The system according to any one of examples 169-176, wherein said ultrasound emissions are characterized by low frequencies.
   Example 178. The system according to any one of examples 169-177, wherein said ultrasound emissions are from about 30 kHz to about 1000 kHz.
   Example 179. The system according to any one of examples 169-178, wherein said ultrasound emissions are emitted for a period of time of from about 5 seconds to about 30 seconds.
   Example 180. The system according to any one of examples 169-179, wherein said ultrasound emissions are emitted for a period of time of from about 3 seconds to about 120 seconds.
   Example 181. The system according to any one of examples 169-180, wherein said ultrasound emissions are emitted for a period of time of more than 2 seconds.
   Example 182. The system according to any one of examples 169-181, wherein said ultrasound emissions are characterized by an intensity range of from about 0.5 Watt/cm2 to about 2.2 Watt/cm2.
   Example 183. The system according to any one of examples 169-182, wherein said ultrasound emissions are characterized by an intensity range of from about 0.1 Watt/cm2 to about 10 Watt/cm2.
   Example 184. The system according to any one of examples 169-183, wherein said implant is printed within a supportive subtract.
   Example 185. The system according to any one of examples 169-184, wherein said printed within said supportive material is performed before implantation of said implant.
   Example 186. The system according to any one of examples 169-185, wherein said printed within said supportive material is performed after implantation of said implant.
   Example 187. The system according to any one of examples 169-186, wherein said supportive material comprises one or more of agar, gelatin and Pluronic F-127.
   Example 188. The system according to any one of examples 169-187, wherein said supportive material is washable away.
   Example 189. The system according to any one of examples 169-188, wherein said implant comprises a dedicated form when focused ultrasound is applied to said implant according to a predetermined CAD model layer.
   Example 190. The system according to any one of examples 169-189, wherein said ultrasound transducers are planar ultrasound transducers.
   Example 191. The system according to any one of examples 169-190, wherein said ultrasound transducers are focused ultrasound transducers.
   Example 192. A method of implanting an implant, comprising:

a. implanting an acoustic-sensitive material comprising a solution of pre-polymer and acoustic-sensitive cross-linker loaded micro-capsules; and in a first site of said patient;

b. selectively hardening said solution by emitting acoustic energy to a second site of said patient.

Example 193. The method according to example 192, wherein said first site and said second site are the same site.
Example 194. The method according to example 192 or example 193, wherein said first site and said second site are different sites.
Example 195. The method according to any one of examples 192-194, wherein said first site is an implantation target site.
Example 196. The method according to any one of examples 192-195, wherein said first site is a blood vessel.
Example 197. The method according to any one of examples 192-196, wherein said second site is said implantation target site.
Example 198. The method according to any one of examples 192-197, wherein said acoustic-sensitive cross-linker loaded micro-capsules comprise liposomes including said cross-linker.
Example 199. The method according to any one of examples 192-198, wherein said pre-polymer comprises alginate.
Example 200. The method according to any one of examples 192-199, wherein said acoustic-sensitive material comprises materials with functional acrylate or diacrylate or methacrylate groups.
Example 201. The method according to any one of examples 192-200, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 202. The method according to any one of examples 192-201, wherein said acoustic-sensitive material further comprises a plurality of cells within said acoustic-sensitive material.
Example 203. The method according to any one of examples 192-202, wherein said acoustic-sensitive material further comprises at least one releasable drug within said acoustic-sensitive material.
Example 204. The method according to any one of examples 192-203, wherein said emitting acoustic energy comprises emitting ultrasound emissions.
Example 205. The method according to any one of examples 192-204, wherein said emitting comprises emitting at low frequencies.
Example 206. The method according to any one of examples 192-205, wherein said emitting comprises emitting at a frequency of from about 30 kHz to about 1000 kHz.
Example 207. The method according to any one of examples 192-206, wherein said emitting comprises emitting for a period of time of from about 5 seconds to about 30 seconds.
Example 208. The method according to any one of examples 192-207, wherein said emitting comprises emitting for a period of time of from about 3 seconds to about 120 seconds.
Example 209. The method according to any one of examples 192-208, wherein said emitting comprises emitting for a period of time of more than 2 seconds.
Example 210. The method according to any one of examples 192-209, wherein said ultrasound emissions are characterized by an intensity range of from about 0.5 Watt/cm2 to about 2.2 Watt/cm2.
Example 211. The method according to any one of examples 192-210, wherein said ultrasound emissions are characterized by an intensity range of from about 0.1 Watt/cm2 to about 10 Watt/cm2.
Example 212. The method according to any one of examples 192-211, wherein said selectively hardening is performed within a supportive subtract.
Example 213. The method according to any one of examples 192-212, wherein said selectively hardening within said supportive material is performed before implantation of said implant.
Example 214. The method according to any one of examples 192-213, wherein said selectively hardening within said supportive material is performed after implantation of said implant.
Example 215. The method according to any one of examples 192-214, wherein said supportive material comprises one or more of agar, gelatin and Pluronic F-127.
Example 216. The method according to any one of examples 192-215, wherein said method further comprises washing away said supportive material.
Example 217. The method according to any one of examples 192-216, wherein said method further comprises providing a dedicated form to said implant by emitting focused ultrasound to said implant according to a predetermined CAD model layer.
Example 218. The method according to any one of examples 192-217, wherein said emitting acoustic energy comprise emitting via planar ultrasound transducers.
Example 219. The method according to any one of examples 192-218, wherein said emitting acoustic energy comprise emitting via focused ultrasound transducers.
Example 220. A method of providing a determined form to an implant inside a body of a patient, comprising:

a. preparing a virtual model of said implant;

b. preparing a solution comprising pre-polymer and acoustic-sensitive cross-linker loaded micro-capsules;

c. injecting said solution at a first location in said body of said patient;

d. selectively hardening said solution according to said virtual model of said implant at a second location in said body of said patient.

Example 221. The method according to example 220, wherein said first site and said second site are the same site.
Example 222. The method according to example 220 or example 221, wherein said first site and said second site are different sites.
Example 223. The method according to any one of examples 220-222, wherein said first site is an implantation target site.
Example 224. The method according to any one of examples 220-223, wherein said first site is a blood vessel.
Example 225. The method according to any one of examples 220-224, wherein said second site is said implantation target site.
Example 226. The method according to any one of examples 220-225, wherein said acoustic-sensitive cross-linker loaded micro-capsules comprise liposomes including said cross-linker.
Example 227. The method according to any one of examples 220-226, wherein said pre-polymer comprises alginate.
Example 228. The method according to any one of examples 220-227, wherein said acoustic-sensitive material comprises materials with functional acrylate or diacrylate or methacrylate groups.
Example 229. The method according to any one of examples 220-228, wherein said acoustic-sensitive material comprises one or more of PEG-DA, PVA-MA, PBS, HAMA, PCL, PLA, PLGA, PBS, Matrigel, PEG-fibrinogen, Collagen, Fibronectin, Hydroxyapatite, alginate, glycerol.
Example 230. The method according to any one of examples 220-229, wherein said acoustic-sensitive material further comprises a plurality of cells within said acoustic-sensitive material.
Example 231. The method according to any one of examples 220-230, wherein said acoustic-sensitive material further comprises at least one releasable drug within said acoustic-sensitive material.
Example 232. The method according to any one of examples 220-231, wherein said emitting acoustic energy comprises emitting ultrasound emissions.
Example 233. The method according to any one of examples 220-232, wherein said emitting comprises emitting at low frequencies.
Example 234. The method according to any one of examples 220-233, wherein said emitting comprises emitting at a frequency of from about 30 kHz to about 1000 kHz.
Example 235. The method according to any one of examples 220-234, wherein said emitting comprises emitting for a period of time of from about 5 seconds to about 30 seconds.
Example 236. The method according to any one of examples 220-235, wherein said emitting comprises emitting for a period of time of from about 3 seconds to about 120 seconds.
Example 237. The method according to any one of examples 220-236, wherein said emitting comprises emitting for a period of time of more than 2 seconds.
Example 238. The method according to any one of examples 220-237, wherein said ultrasound emissions are characterized by an intensity range of from about 0.5 Watt/cm2 to about 2.2 Watt/cm2.
Example 239. The method according to any one of examples 220-238, wherein said ultrasound emissions are characterized by an intensity range of from about 0.1 Watt/cm2 to about 10 Watt/cm2.
Example 240. The method according to any one of examples 220-239, wherein said selectively hardening is performed within a supportive subtract.
Example 241. The method according to any one of examples 220-240, wherein said selectively hardening within said supportive material is performed before implantation of said implant.
Example 242. The method according to any one of examples 220-241, wherein said selectively hardening within said supportive material is performed after implantation of said implant.
Example 243. The method according to any one of examples 220-242, wherein said supportive material comprises one or more of agar, gelatin and Pluronic F-127.
Example 244. The method according to any one of examples 220-243, wherein said method further comprises washing away said supportive material.
Example 245. The method according to any one of examples 220-244, wherein said method further comprises providing a dedicated form to said implant by emitting focused ultrasound to said implant according to a predetermined CAD model layer.
Example 246. The method according to any one of examples 220-245, wherein said emitting acoustic energy comprise emitting via planar ultrasound transducers.
Example 247. The method according to any one of examples 220-246, wherein said emitting acoustic energy comprise emitting via focused ultrasound transducers.
Example 248. A method of generating an acoustic-sensitive implant comprising at least one cell, comprising:

a. adding said at least one cell into a hydrogel solution thereby generating a cell/hydrogel solution;

b. contemporarily injecting said cell/hydrogel solution and at least one oil via a dedicated syringe, thereby generating individual cell/hydrogel beads;

c. dropping said individual cell/hydrogel beads in a calcium chloride solution;

d. separating said individual cell/hydrogel beads from said calcium chloride solution;

e. adding said separated individual cell/hydrogel beads into a PEG-DA solution;

Example 249. The method according to example 248, wherein said hydrogel is alginate.
Example 250. The method according to example 248, wherein said hydrogel is one or more of fibrin and gelatin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and/or images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A (i-iv) are exemplary stages of rapid polymerization of acoustic-sensitive PEG-DA based material, according to some embodiments of the invention;

FIG. 1B is a representative image of hydrogel polymerization post US induction at 37 kHz, according to some embodiments of the invention;

FIG. 2A (i-iii) are exemplary bulk hydrogel formations under 5, 10 and 30 second of 1 MHz US exposure, respectively, according to some embodiments of the invention;

FIG. 2B is a representative image of an exemplary rheometric system used for mechanical properties characterization of the US induced hydrogels, according to some embodiments of the invention;

FIG. 2C is a graph comparing exemplary young modulus of US induced bulk hydrogels for various exposure times, according to some embodiments of the invention;

FIG. 2D is a graph showing exemplary US induced hydrogel conversion rate for various exposure times, according to some embodiments of the invention;

FIG. 2E (i-ii) are exemplary SEM images of US induced hydrogels formed under 20 sec (i) or 30 sec (ii) US exposure time, according to some embodiments of the invention;

FIGS. 2F, 2G, 2H, 2I, 2J and 2K are images showing exemplary mechanical characterization properties, according to some embodiments of the invention;

FIG. 3A is a US mediated polymerization through 6 cm breast phantom, according to some embodiments of the invention;

FIG. 3B (i-ii) show US mediated polymerization through 2 cm bovine brain tissue (3Bi), and the resulted hydrogel (3Bii), according to some embodiments of the invention;

FIG. 3C (i-ii) show US mediated polymerization through 4 cm bovine muscle tissue (3Ci), and the resulted hydrogel (3Cii), according to some embodiments of the invention;

FIG. 4A shows polymerized hydrogel with living cells (DPSCs) for cell delivery applications, according to some embodiments of the invention;

FIG. 4B (i-ii) are representative images of DPSCs cell within the hydrogel post US polymerization, according to some embodiments of the invention;

FIG. 4C shows different exemplary materials exposed to about 30 sec US induction and solidified into hydrogel bulks, according to some embodiments of the invention;

FIGS. 4D, 4E, 4F, 4G, 4H and 4I are images showing exemplary optimization of an exemplary cell delivery protocol, according to some embodiments of the invention

FIG. 5A shows an exemplary solidified drug loaded bulk that serves as a device for in situ sustain drug release, according to some embodiments of the invention;

FIG. 5B shows an exemplary analysis using Bradford assay of the released levels of BSA, according to some embodiments of the invention;

FIG. 5C shows a graph of the release of BSA over time in the samples, according to some embodiments of the invention;

FIGS. 5D and 5E are exemplary optimization of ultrasound mediated polymerization for drug delivery, according to some embodiments of the invention;

FIG. 6A (i-iii) show exemplary steps for 3D printing, bioprinting and spatial templating of acoustic-sensitive materials, according to some embodiments of the invention;

FIG. 6B (i-ii) show exemplary printed objects, according to some embodiments of the invention;

FIG. 7A (i-ii) are exemplary adjusted viscous materials subjected to US induction that results in polymerization, according to some embodiments of the invention;

FIG. 7B (i-ii) are exemplary localized polymerization of the acoustic-sensitive viscous material by using a conic tube with a convex bottom, according to some embodiments of the invention;

FIG. 8A is an image of an exemplary concentrated calcium loaded liposomes during an exemplary preparation protocol, according to some embodiments of the invention;

FIG. 8B (i-ii) show fluorescent microscopy images of exemplary calcium loaded liposomes labeled with fluorescein for 3D alginate printing using FUS for local calcium release, according to some embodiments of the invention;

FIG. 9 is a flowchart of exemplary methods, according to some embodiments of the invention; and

FIGS. 10A-10B are exemplary PVA-MA material used, according to some embodiments of the invention.

### DESCRIPTION OF SPECIFIC EMBODIMENTS OF THE INVENTION

The present invention, in some embodiments thereof, relates to polymerization of implants and, more particularly, but not exclusively, to a polymerization of implants inside the body with optionally performing an additional action.

### Overview

An aspect of some embodiments of the invention relates to in-situ bioprinting based on ultrasound (US) mediated polymerization in which the triggering acoustic system is applied completely external to the patient and enables non-invasive spatial templating of 3D implants, in-situ, from an injected acoustic-sensitive material. In some embodiments, the injected material serves as a bridging medium for deep tissue defects and/or as a localized controlled and sustained drug delivery scaffold by combining a drug with the injected acoustic-sensitive material. In some embodiments, by combining tissue-specific and/or therapeutic cells with the injected material, the invention serves as a platform for cell-based therapies for tissue regeneration and augmentation. In some embodiments, potential advantages of utilizing ultrasound printing and polymerization are: ultrasound is a non-invasive method of performing printing and/or polymerization at any location inside the patient, including all deep tissues and internal organs, with the material being transported to the area in a single injection. In some embodiments, contrary to extrusion printing that are performed on the surface only (skin) and/or by performing very invasive procedures, the present invention allows full exposure access and maneuvering of the “printing head” in the three axes within the patient's body (x, y, z). In some embodiments, another potential advantage is that ultrasound procedures are much safer for the patient, are less likely to damage blood vessels and nerves during the procedure and are less risky of developing postoperative infections. In some embodiments, another potential advantage of the invention is that utilizing external ultrasound requires less preparation time and much shorter overall operation time than the preparations required for invasive procedures that require to expose the treatment area and insert the “print head” and all the accompanying equipment into the patient, which additionally potentially makes the ultrasound treatment much more economical. In some embodiments, the biocompatible materials do not require photoinitiators, which are toxic to cells, which are required by the method of printing in extrusion and polymerization in light. In some embodiments, the printed material comprises living cells for implantation. In some embodiments, the printed material with living cells does not heat up, therefore potentially avoiding damaging the cells and/or the tissues of the patient with heating. In some embodiments, at least 95% of the polymerization is performed in a few seconds, for example from about 5 seconds to about 10 seconds. Optionally from about 4 seconds to about 30 seconds. Optionally from about 3 seconds to about 120 seconds.

In some embodiments, the ultrasound used for polymerization is a low power therapeutic and optionally FDA approved ultrasound (focused or non-focused). In some embodiments, a potential advantage of utilizing low power US is that it potentially increases the survival of the implanted cells and potentially prevents damage to tissues.

In some embodiments, the implants comprise one or more material compositions (for example PEGDA), which optionally also contain ECM proteins that potentially allow to provide implanted cells with higher survival rates and higher functionality performance.

In some embodiments, the implant is configured for controlled release of drugs (for example by local injection or blood circulation), polymerization by general ultrasound induction of the implant (with or without cells) in deep tissues for reconstruction and regeneration.

An aspect of some embodiments of the invention relates to 3D printing in the body of a patient with a low-power external US, where the polymerization effect is through cavitation and not thermal polymerization.

In some embodiments, 3D printing in the body is performed in deep locations in the body, contrary to current technologies, which allow only printing on the surface (skin) or up to 1 mm into the skin using near infrared light (NIR). In some embodiments, polymerization levels are controlled by changing ultrasound exposure time and/or intensity, therefore allowing to control the stiffness of the implant according to treated tissue and/or controlling drug release profile over-time as needed.

In some embodiments, a potential advantage of the invention is that it provides a noninvasive 3D bioprinting and/or cell delivery and/or drug delivery within deep tissues or inner organs which potentially enables to perform safer procedures.

In some embodiments, another potential advantage of the invention is that the acoustic mediated polymerization is initiators-free which makes the acoustic-sensitive material more biocompatible as compared to other printing methods, which use toxic initiators that can damage the delivered cells or the surrounding tissues. In some embodiments, another potential advantage is the reduction of the procedure time as compared to traditional invasive procedures.

In some embodiments, injection of the pre-polymerized implant is performed at the location where the implant is wanted or via the blood vessels. In some embodiments, ultrasound is applied at the location where the implant is wanted, no matter the place of delivery (blood vessel or locally). In some embodiments, the ultrasound is performed using planar transducers when a specific form of the implant is not needed, and focused ultrasound is used when a specific form is required.

Before explaining at least one embodiment of the invention in detail, it is to be understood that the invention is not necessarily limited in its application to the details set forth in the following description or exemplified by the Examples and/or the details of construction and the arrangement of the components and/or methods set forth in the following description and/or illustrated in the drawings and/or the Examples. The invention is capable of other embodiments or of being practiced or carried out in various ways.

Exemplary General Information about the Invention

In some embodiments, ultrasound-mediated polymerization for cell and drug delivery procedure comprises the following actions:

Preparing of the acoustic-sensitive materials: these materials comprise one or more and not limited to PEG-DA, PBS, Matrigel, PEG-fibrinogen, Hydroxyapatite, alginate, glycerol (in some embodiments can include other materials with functional diacrylate or methacrylate groups).

Applying ultrasound induction using low frequency transducers (from about 30 kHz to about 1000 kHz) for from about 5 seconds to about 30 seconds in order to reach polymerization with an intensity range of from about 0.5 Watt/cm to about 2.2 Watt/cm.

In some embodiments, for drug delivery applications, the chosen drug concentration is loaded into an acoustic-sensitive mixture before the application of ultrasound.

In some embodiments, for cell delivery application, cell pellets are added to the acoustic-sensitive mixture briefly before the application of ultrasound.

In some embodiments, for ultrasound printing within supportive substrate, a self-healing 3D supportive substrate is prepared in advanced using one or more of agar, gelatin, Pluronic F-127, or other support media followed by 3D printing of the acoustic-sensitive mixture in the desired template and followed by ultrasound application for polymerization. In some embodiments, right after the ultrasound application, the support material is washed away, and the printed sample is extracted.

In some embodiments, for in-situ noninvasive printing the acoustic-sensitive mixture is injected into the area of interest followed with local patterning by applying focused ultrasound induction according to the CAD model, layer by layer, until polymerizing the full model shape. In some embodiments, the residual unpolymerized material is then cleared from the treated area using a minimally invasive collecting needle.

### Exemplary Applications

In some embodiments, the invention is used for noninvasive in-situ patterning/bioprinting of cellular/acellular scaffolds for tissue reconstruction (such as bone, cartilage, muscle, soft tissues etc.). In some embodiments, scaffold stiffness can be modified to fit the target tissue by changing material composition and time exposure to ultrasound induction, for example Hydroxyapatite or Polycaprolactone or Polylactic acid or nanosilicates can be added to the mixture to increase the stiffness. In some embodiments, scaffold stiffness is from about 1 kPa to about 100 kPa. Optionally from about 0.5 kPa to about 250 kPa. Optionally from about 0.1 kPa to about 500 kPa.

In some embodiments, the invention is used for local and deep polymerization for sustained drug release using local injection or intravenous infusion and local polymerization in the sight of interest. In some embodiments, the release profile over-time can be modified by changing acoustic-sensitive material properties during polymerization protocol.

### Exemplary Rapid Polymerization by Ultrasound Induction

In some embodiments, in order to achieve initiators free, rapid polymerization of an acoustic-sensitive material, the following exemplary protocol is used:

In some embodiments, fresh 10% PEG-DA/PBS mixture having a pH 7.5, is exposed to various US induction profiles, for example, about 37 kHz low frequency and about 1 MHz high frequency with various induction periods from about 5 seconds to about 60 seconds and a signal power of from about 0.3 Watt/cm\hat{ }2 to about 2.2 Watt/cm\hat{ }2.

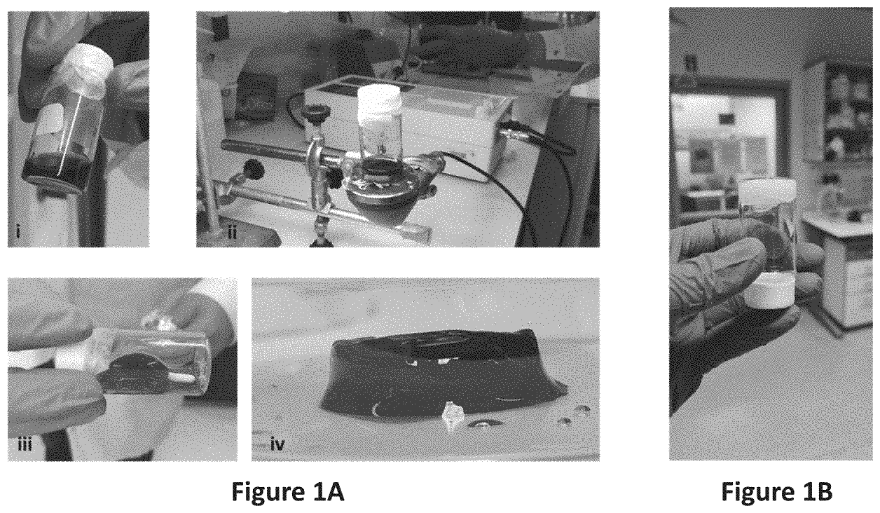

In some embodiments, the described conditions leads to bulk hydrogel formation from liquid pre-polymer solution, as can be seen in FIG. 1A (i-iv) and FIG. 1B. FIG. 1A shows a rapid polymerization of acoustic-sensitive PEG-DA based material—before induction (1Ai), during 1 MHz US induction (1Aii), and post polymerization (1Aiii and 1Aiv). FIG. 1B shows a representative image of hydrogel polymerization post US induction at 37 kHz. In some embodiments, the polymerization mechanism is based on cavitation formation followed by radical formation (OH—) and covalent bonding of the polymer chains via the functional groups (Acrylate/Methacrylate). In some embodiments, the minimal US induction period needed to achieve bulk hydrogel polymerization is about 5 seconds, and the minimal US power needed for bulk hydrogelation is as low as about 0.3 Watt/cm\hat{ }2. In some embodiments, the polymer chains bond covalently and the formed hydrogel stays stable for long time periods (months or more).

### Exemplary Tuning of Mechanical Properties by US Induction Profile

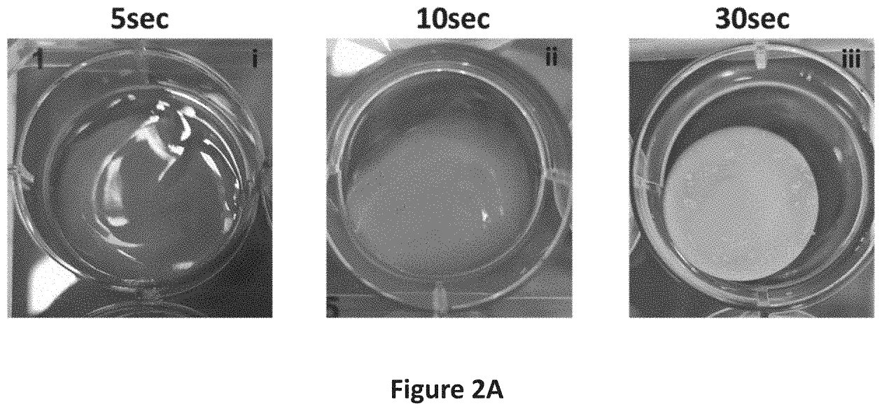

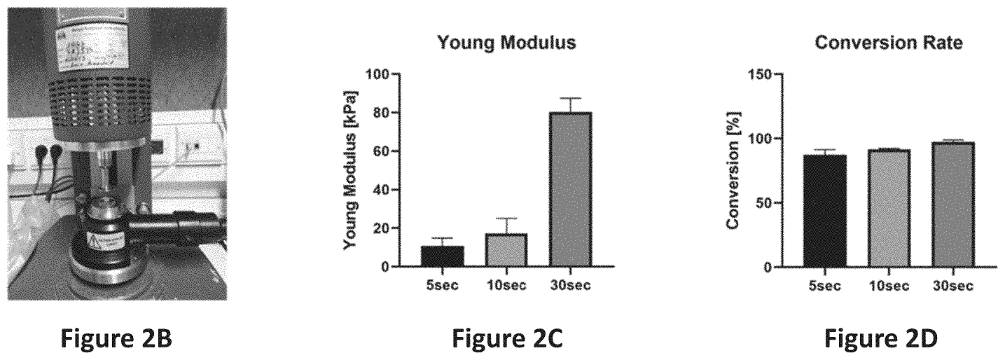

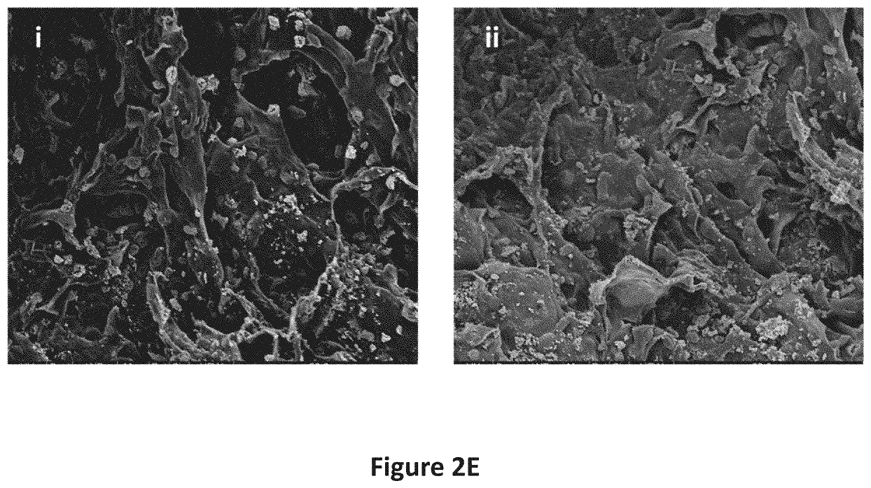

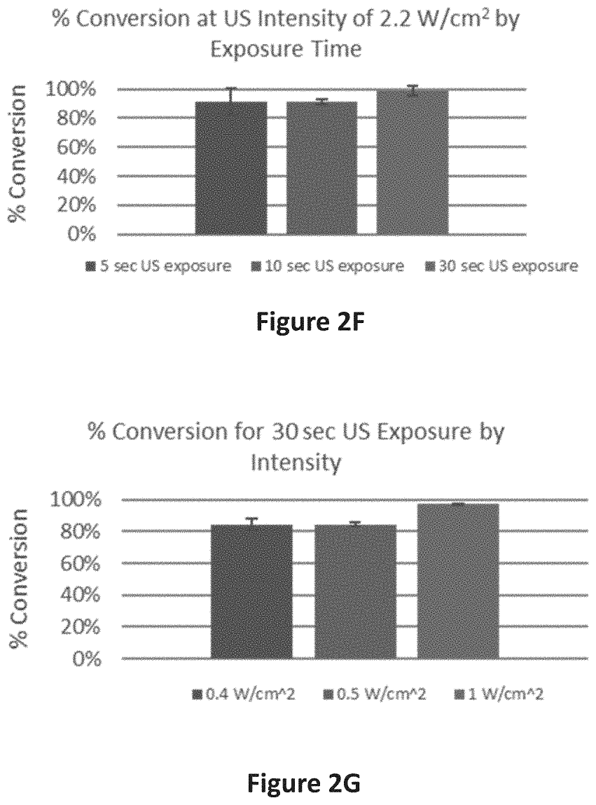

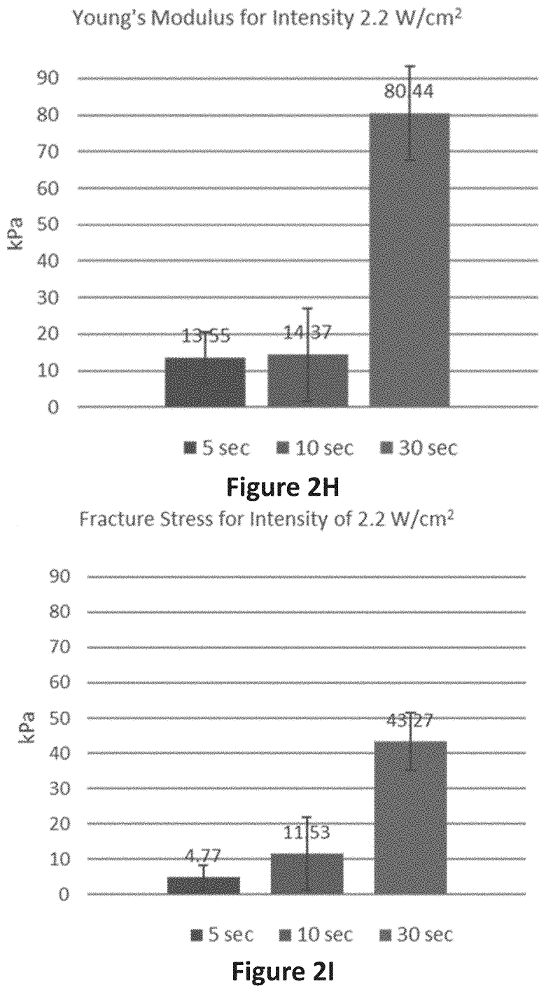

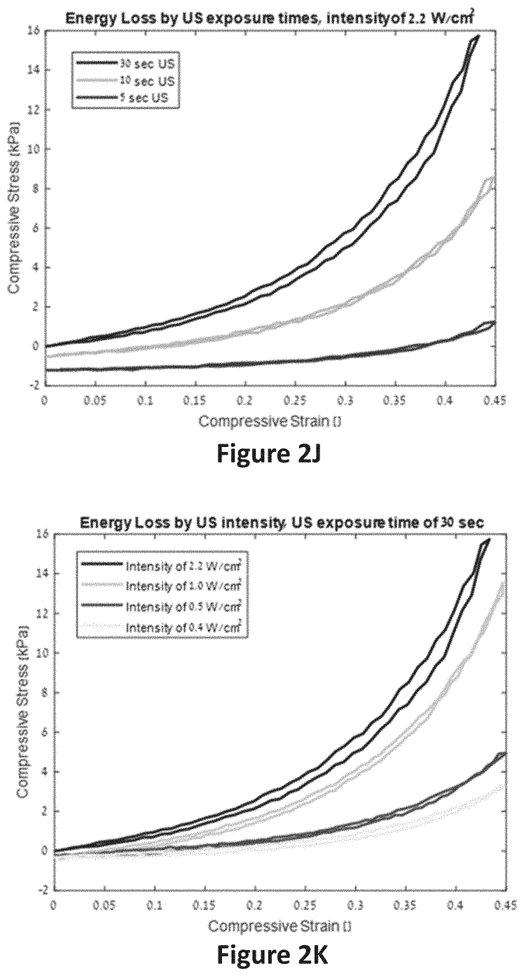

In some embodiments, the mechanical properties of the polymerized hydrogels formed by various US induction periods including 5, 10 and 30 seconds are measured and analyzed. In some embodiments, the conversion rates in all of the tested groups reached high percentage (from about 87% to about 97%) indicating very fast and efficient polymerization reaction, as shown for example in FIG. 2A (i-iii), FIG. 2B, FIG. 2C and FIG. 2D. FIG. 2A (i-iii) shows bulk hydrogel formation under 5, 10 and 30 second of 1 MHz US exposure, respectively. FIG. 2B shows a representative image of the rheometric system used for mechanical properties characterization of the US induced hydrogels. FIG. 2C shows a graph comparing young modulus of US induced bulk hydrogels for various exposure times. FIG. 2D shows exemplary US induced hydrogen conversion rate for various exposure times. In some embodiments, the rigidity of the hydrogel is controlled by adjusting the US induction periods as can be observed by the wide range of the measured young modulus values for different induction periods, as shown in FIGS. 2B and 2C, as well as the formed micro-structure which showed higher pore size at shorter induction periods, as shown for example in FIG. 2E (i-ii). FIG. 2E (i-ii) show exemplary SEM images of US induced hydrogels formed under 20 sec (i) or 30 sec (ii) US exposure time. In some embodiments, a potential advantage of this feature is that it can be leveraged in variety of potential in-vivo applications in which the mechanical properties of the delivered US induced hydrogel are adjusted to match the rigidity of the target tissue (for example low rigidity for soft tissues like the brain, high rigidity for harder tissues like cartilage or bone).

Referring now to FIGS. 2F-2K, showing exemplary mechanical characterization properties, according to some embodiments of the invention. In some embodiments, mechanical properties characterization for the bulk hydrogels polymerized by various US induction profiles we performed. In some embodiments, mechanical characterization includes measuring the material conversion rate, material stiffness (Young's modulus values), material fracture stress, energy loss under cyclic loading and material microstructure characterization.

### Exemplary Polymer Conversion Rate in Mechanical Characterizations

In some embodiments, in order to assess the polymer conversion rate, glass vials with 4 ml of the 10% PEG-DA solution were exposed to a 1 MHz US probe. In some embodiments, 18 samples were exposed to US set at the intensity of 2.2 W/cm² for 5, 10 and 30 seconds, 6 samples for each exposure time. In some embodiments, additionally, 12 samples were exposed to US for 30 seconds at intensities of 0.4, 0.5 and 1 W/cm², 4 samples for each intensity. In some embodiments, in each case the percent conversion was calculated by measuring the amount of hydrogel that remained liquid and did not become part of the polymerized gel, as shown for example in the graphs showed in FIG. 2F and FIG. 2G. In some embodiments, the hydrogel exposed to the US for 30 seconds showed the highest conversion rate reaching 98.9% (FIG. 2F). In some embodiments, the hydrogel exposed to high intensity US (2.2 W/cm2) showed the highest conversion rate—97%, but that is still lower than the 30 sec, 2.2 W/cm2 rate seen in FIGS. 2F and 2G. In some embodiments, the conversion graphs show that almost all the fluid is converted to the gel form, indicating a high efficiency of the polymerization process.

### Exemplary Mechanical Properties Measured by Rheometer

In some embodiments, in order to assess the mechanical properties measured by Rheometer, 8 mm diameter hydrogel slices were loaded onto a Rheometer plate fitted with a 20 mm parallel plate geometry to perform cyclic uniaxial compression tests. In some embodiments, the samples underwent 3 rounds of compression and relaxation reaching a maximal strain of 45%. In some embodiments, in a 4^{th} round, pressure was applied reaching the fracture point of the hydrogel. In some embodiments, this protocol was performed for PEG-DA hydrogels polymerized using US intensity of 2.2 W/cm² for 5, 10 or 30 seconds. In some embodiments, 4 samples were measured for each of the exposure times. In some embodiments, fracture stress was calculated, as well as Young's modulus, for multiple US exposure times and intensities. Young's modulus was calculated by linearization of the stress-strain curve in the range of 35%-45% strain. In some embodiments, there is a direct correlation between the US exposure time and the stiffness of the hydrogel, as can be seen for example in FIG. 2H. In some embodiments, the Young's modulus after US exposure measured 13.55, 14.37 and 80.44 kPa after 5, 10 and 30 seconds respectively. In some embodiments, the stress under which the hydrogels fractured were 4.77, 11.53 and 43.27 kPa for US exposure times of 5, 10 and 30 seconds respectively, as shown for example in FIG. 2I. In some embodiments, these results indicate the increase in stiffness in correlation with US exposure time. In some embodiments, FIG. 2J shows the loss of energy during one cycle of compression and relaxation of the hydrogels after US exposure. Each curve reaches a maximum during compression and returns to the starting point during the relaxation at a greater slope. In some embodiments, this difference creates a gap between the two stress curves. In some embodiments, the area between the two curves (the gap) represents the energy lost. In some embodiments, the more energy lost, the less resilient the material. In some embodiments, in FIG. 2J the gap is greatest for the maximum time exposure of 30 seconds and in FIG. 2K the gap is greatest for the maximum intensity exposure of 2.2 W/cm². These results match the mechanical properties shown in FIGS. 2H and 21 showing that the higher or longer the US exposure, the stiffer and less resilient the hydrogel.

### Exemplary US Mediated Polymerization Through Thick Tissues

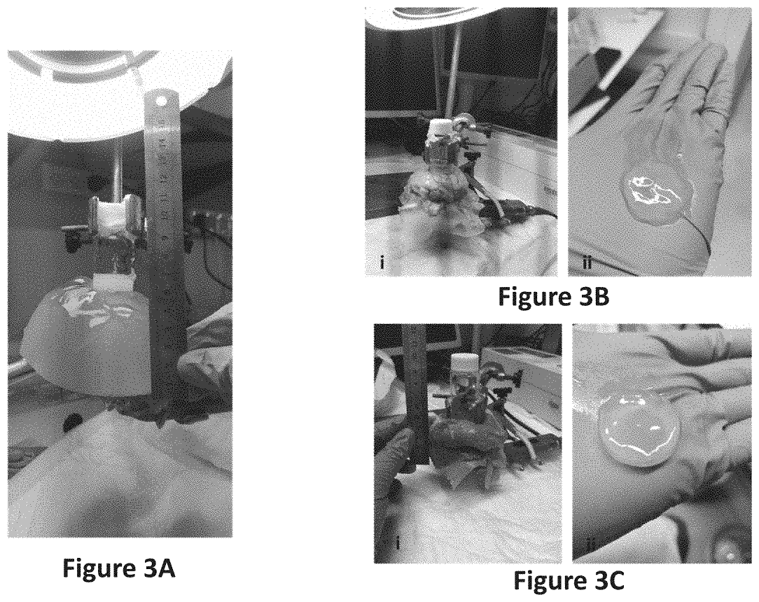

In some embodiments, several experiments for inducing polymerization through very thick and dense tissues and implants were performed to test the feasibility of inducing polymerization deep in the patient body by using external US transducer/probe. The results of those experiments can be seen in FIGS. 3A, 3B (i-ii) and 3C (i-ii). FIG. 3A shows a US mediated polymerization through 6 cm breast phantom. The US transducer located under the phantom bottom and the acoustic-sensitive material located on the top. FIG. 3B (i-ii) show US mediated polymerization through 2 cm bovine brain tissue (3Bi), and the resulted hydrogel (3Bii). FIG. 3C (i-ii) shows US mediated polymerization through 4 cm bovine muscle tissue (3Ci), and the resulted hydrogel (3Cii). In some embodiments, as can be seen in FIGS. 3A, 3B (i-ii) and 3C (i-ii), the induction of US (1 MHz) for 30 sec leads to complete polymerization through 6-centimeter breast phantom (FIG. 3A), through 2 centimeter of bovine brain tissue (FIG. 3B (i-ii)), and through 4-centimeter bovine muscle tissue (FIG. 3C (i-ii)). In some embodiments, these results highlight the ability of the US based technology to penetrate non-invasively deep into the patient body and induce full polymerization rapidly (seconds) and safely (low intensity).

Exemplary US Mediated Polymerization of Biocompatible Materials with or without Cells for Cell and Scaffold Delivery

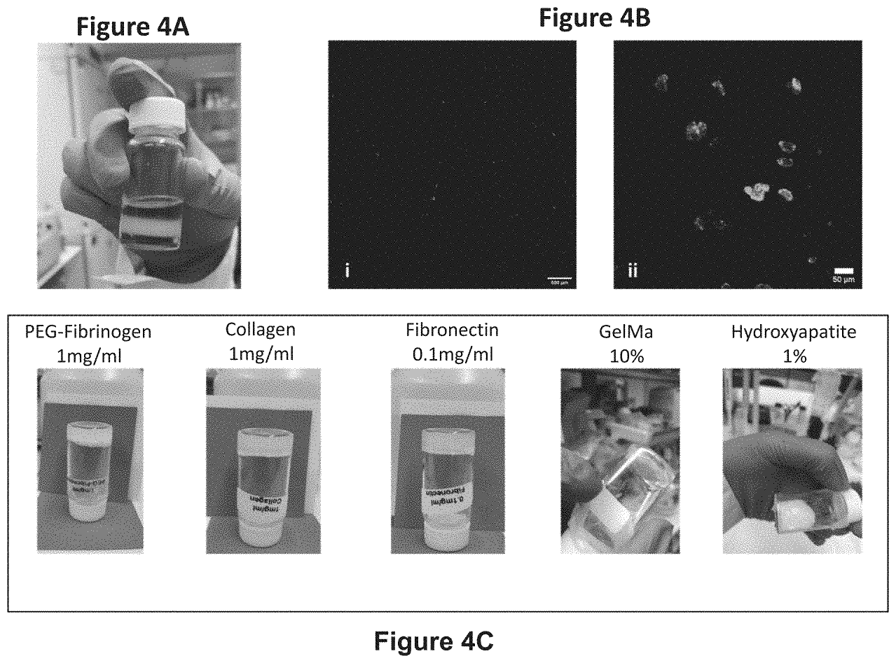

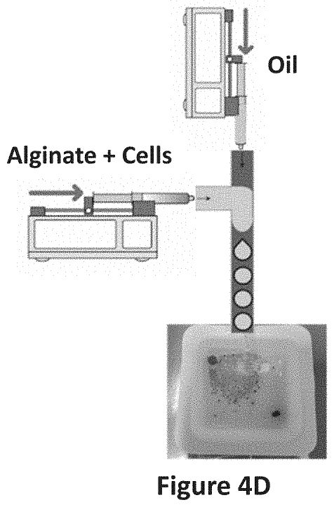

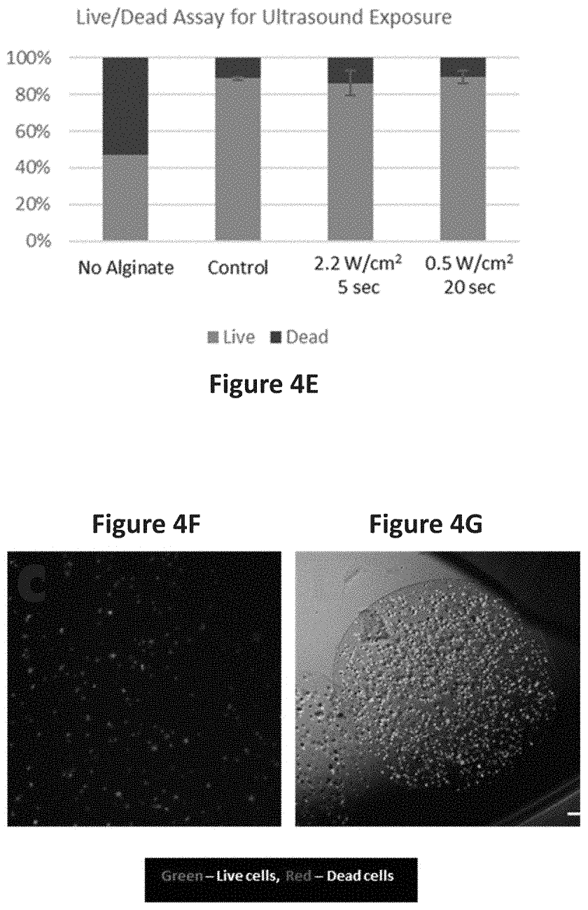

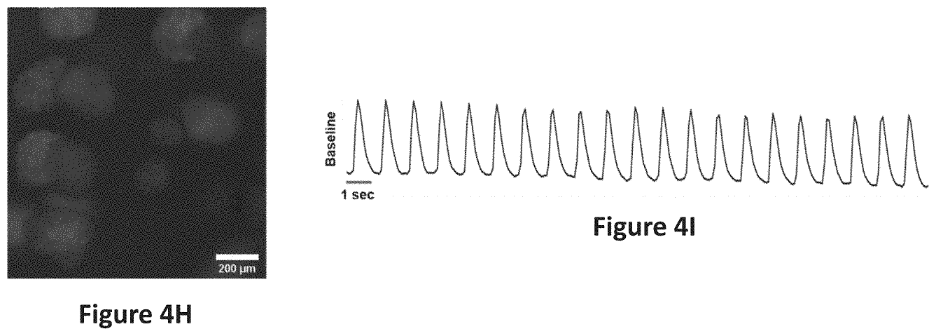

In some embodiments, for cell delivery applications, FIGS. 4A and 4B (i-ii) show both acoustic-sensitive material polymerization with living cells by US induction and the ability to induce polymerization by US on a variety of biocompatible materials and mixtures containing ECM proteins for supporting cell functionality post-delivery. FIG. 4A shows polymerized hydrogel with living cells (DPSCs) for cell delivery applications. The acoustic-sensitive solution mixed with DPSCs cells and the mixture was polymerized by low intensity rapid US induction and cultivated with DPSCs medium post polymerization. FIG. 4B (i-ii) are representative images of DPSCs cell within the hydrogel post US polymerization. In some embodiments, the materials compositions were exposed to about 30 sec US induction and solidified into hydrogel bulks, as shown in FIG. 4C from left to right—PEG-DA, PEG-fibrinogen, the combination of PEG-DA with collagen, fibronectin, GelMa, Hydroxyapatite (bone mineral). In some embodiments, these biocompatible materials are injected with/without cells to the treated tissue in liquid state, solidified by external US application and potentially induce tissue regeneration by the deposition of pro-regenerative scaffold or therapeutic cell delivery.

Referring now to FIGS. 4D-4I showing an exemplary optimization of an exemplary cell delivery protocol, according to some embodiments of the invention. In some embodiments, living cells (dental pulp stem cells (DPSCs), fibroblasts and iPSC-derived human cardiomyocytes) were loaded with the acoustic-sensitive materials and polymerization was induced by an external US transducer. In some embodiments, cell viability rate was examined using live/dead staining assay which resulted in about 50% viability rate, as shown for example in FIG. 4E. In some embodiments, in order to improve cell viability, a new protocol was developed in which the delivered cells were encapsulated in a protective alginate microbeads which were loaded into the acoustic-sensitive material. In some embodiments, a potential advantage of encapsulating the cells is that it revealed significantly higher cell viability rates after US induced polymerization (about 90% viability).

### Exemplary Micro Fluid Technique for Cell Encapsulation

In some embodiments, for assessing an exemplary microfluidic technique for cell encapsulation, dental pulp stem cells (DPSCs) or human iPSC-derived cardiomyocytes were added to a 0.5% alginate solution diluted in PBS. In some embodiments, any protective hydrogel can be used instead of alginate, for example fibrin, gelatin, etc. In some embodiments, a dedicated system was set up comprising two syringe pumps where the fluids exiting each syringe met at a 90° angle in a T-junction, as shown for example in FIG. 4D. In some embodiments, corn oil was ejected from one syringe in a vertical position at the same time as the alginate DPSC solution was ejected from the second syringe in a horizontal position. In some embodiments, any kind of oil can be used instead of corn oil. In some embodiments, the two liquids met at the T-junction and individual beads were formed. In some embodiments, the alginate beads were then dropped into a calcium chloride solution in order to allow initiation of the cross-linking and to allow to form stable hydrogel beads. In some embodiments, the beads containing DPSCs formed by micro fluid technique were separated from the calcium chloride solution and were mixed in with the PEG-DA solution for the polymerization protocol. In some embodiments, a standard protocol was performed including argon gas and US polymerization as detailed elsewhere herein for 10 samples. In some embodiments, 5 samples were exposed to US at 2.2 W/cm² for 5 seconds and 5 samples were exposed to US at 0.5 W/cm² for 20 seconds. In some embodiments, additionally, 4 control vials were prepared without undergoing the argon treatment or US exposure. In some embodiments, after US exposure, a live/dead staining assay was performed according to the manufacturer's instructions. In some embodiments, images were then taken using a confocal microscope and the percent of viable cells was calculated. In some embodiments, as shown for example in FIG. 4E and FIG. 4F, with no alginate protection the cell viability is less than 50%. In some embodiments, a potential cause of this is because of the mechanical disturbances caused by the US waves, the free radicals released by cross-linking, and the increased temperature, expected to reduce cell viability. In some embodiments, therefore, in a follow-up experiment, cell viability was achieved by encapsulating the DPSCs in beads of alginate, using a micro-fluid encapsulation technique. In some embodiments, a potential advantage of the encapsulation is that it potentially provides protection by significantly reducing these adverse effects, providing high cell viability, as shown for example in FIG. 4E and FIG. 4G. In some embodiments, this protection is due both to mechanical as well as chemical protection due to the high stiffness and the presence of hydroxyl groups in the alginate. In some embodiments, for assessing the ability of this protocol to support parenchymal cell viability and functionality, human iPSC-derived cardiomyocyte spheroids were encapsulated in 0.5% alginate beads before adding to the 10% PEG-DA solution. In some embodiments, ultrasound induced polymerization protocol was performed according to the standard protocol (0.5 W/cm² intensity for 30 sec) and cell seeded hydrogels were cultivated. In some embodiments, to assess the iPSC-derived cardiomyocytes functionality, the beating profiles were measured by capturing GCaMP-CMs intracellular Ca⁺² transients over time indicating normal BPM, contraction, and decay time, as shown for example in FIG. 4H and FIG. 4I.

### Exemplary US Mediated Polymerization for Sustained Drug Release

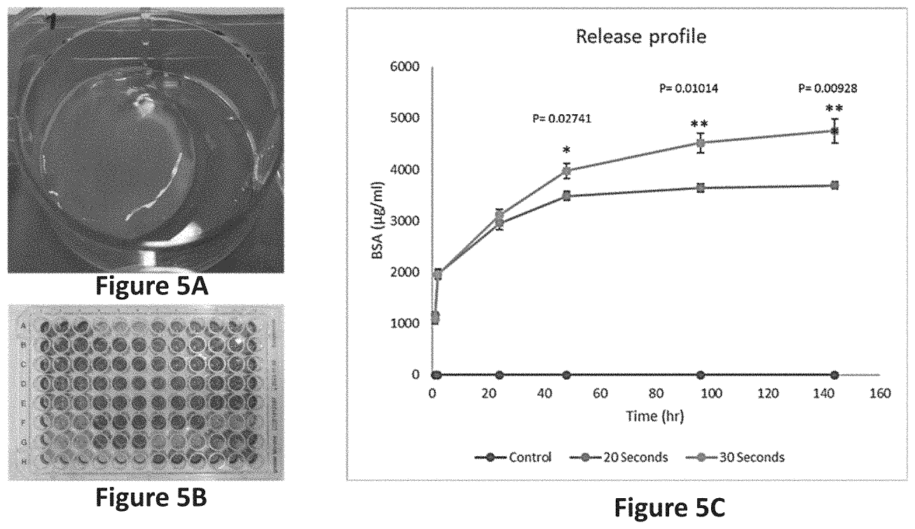

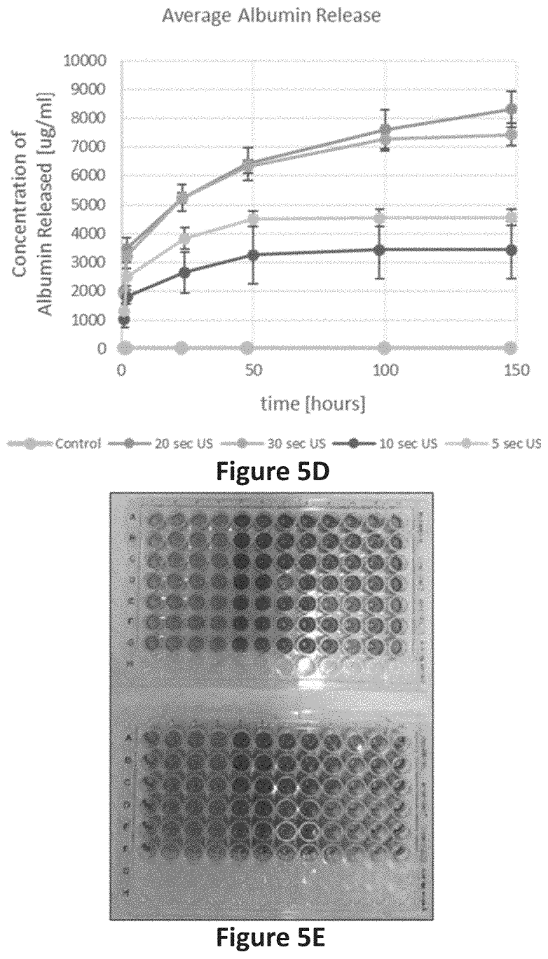

In some embodiments, another application is the ability to deliver, in minimal invasive approach (direct injection to treat site, or by delivering catheter, or intravenously), a pre-polymer with drug solution to the treated site and to induce polymerization by external US transducer. In some embodiments, the solidified drug loaded bulk serves as a device for in situ sustain drug release. In some embodiments, this application was tested using BSA drug release assay in which 1% of BSA was loaded in 10% PEG-DA solution, further solidified by US induction (As shown for example in FIG. 5A) and cultivated in PBS at 37° C. In some embodiments, samples were collected during the following week and the release profiles were analyzed using Bradford assay (As shown for example in FIG. 5B) and the BSA concentration graphs over time were calculated (As shown for example in FIG. 5C). As can be noticed, sustained BSA release was measured during the entire week and the release profiles can be modified by changing the US induction time (30 sec induction time shows slower release profile as compared to 20 sec US induction).

Referring now to FIGS. 5D-5E, showing exemplary optimization of ultrasound mediated polymerization for drug delivery, according to some embodiments of the invention. In some embodiments, in order to assess the optimization for the drug delivery application, a drug release profile was built. In some embodiments, bovine serum albumin (BSA) was used as an example of a molecule representing a drug to be released locally from a hydrogel. In some embodiments, the basic polymerization protocol was used, adding BSA to the PEG-DA solution to reach 1% concentration. In some embodiments, 16 hydrogels were polymerized at US exposure intensity of 2.2 W/cm² for 5, 10, 20 and 30 seconds, 4 hydrogels each. In some embodiments, after polymerization, the hydrogels were placed in PBS solution. In some embodiments, at specific times, 3 samples of the PBS containing the released albumin were taken from each hydrogel well. In some embodiments, fresh PBS was then added, replacing the PBS with released albumin solution. In some embodiments, after one week, using a Bradford assay, the concentration of the released BSA was measured. In some embodiments, as shown for example in FIG. 5D and FIG. 5E, differences in albumin release rates by US exposure time of PEG-DA. In some embodiments, these different profiles are useful for drug release applications. In some embodiments, the hydrogels polymerized by 5 and 10 seconds of US exposure reached a plateau after 96 hours of albumin release whereas the hydrogels polymerized by 20 and 30 seconds of US exposure continued to release albumin throughout the experiment.

### Exemplary US Mediated Polymerization for 3D Printing/Bioprinting

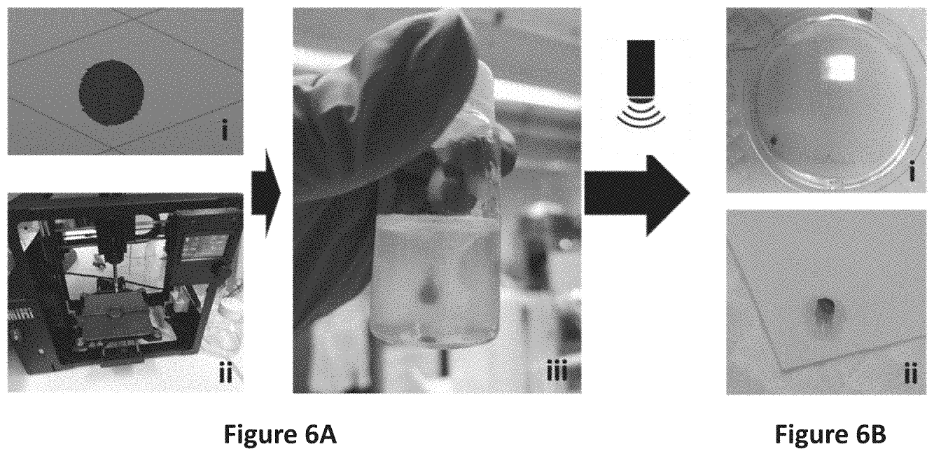

In some embodiments, in another application of the present invention, the application of US induced polymerization was used for 3D printing, bioprinting and spatial templating of acoustic-sensitive materials. In some embodiments, this method is used for fabricating, with or without living cells, pre-designed 3D scaffolds, tissues, organs, drug delivery devices or any other 3D object inside or outside the patient body. In some embodiments, one exemplary printing method, that is conducted inside or outside the body, includes the design of the printed object, as exemplary shown in FIG. 6A (i-iii), 3D printing of the acoustic-sensitive material (with or without cells/drugs) layer by layer within a sacrificial support substrate, followed by general US induction to bulk polymerize the entire printed object, as shown for example in FIG. 6B (i-ii). In some embodiments, another approach is to induce US polymerization for each printed layer separately. In some embodiments, once the printed object is polymerized, the sacrificial support material is removed to release the completed construct (As shown for example in FIG. 6B (i-ii)).

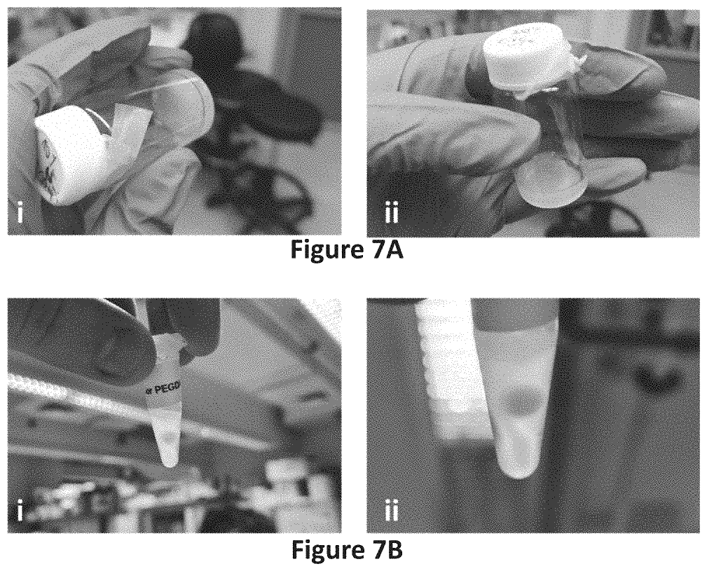

In some embodiments, another exemplary method uses focused ultrasound (FUS) for 3D printing/bioprinting (for all of the applications mentioned above, inside or outside the patient body). In some embodiments, FUS is used to induce polymerization of the acoustic-sensitive material at a localized volume (using for example a cavitation mechanism), and/or by controlling the spatial position of the US focal point the object is printed layer by layer and/or in any other desired pattern. In some embodiments, for 3D bioprinting inside the patient, the acoustic-sensitive visco-elastic material is delivered into the target area (for example by minimally invasive procedure as injection, catheter etc.) and the object is printed in-situ while the unpolymerized material is later cleared away by a collecting needle. In some embodiments, the properties of the acoustic-sensitive material are adjusted to achieve the ability to induce local polymerization and to decrease the diffusion rate of the liquid solution. In some embodiments, for this purpose, materials comprising higher viscosity are used, for example by including alginate, glycerol, or Pluronic-F127 to the PEG-DA solution. In some embodiments, the adjusted viscous materials are subjected to US induction that results in polymerization, as is shown for example in FIG. 7A (i-ii). In some embodiments, in addition, by using a conic tube with a convex bottom, the US waves are concentrated into a spatial focal point from where it can be observed a localized polymerization of the acoustic-sensitive viscous material, as shown for example in FIG. 7B (i-ii).

### Exemplary Calcium Encapsulation for US Mediated Polymerization of Alginate

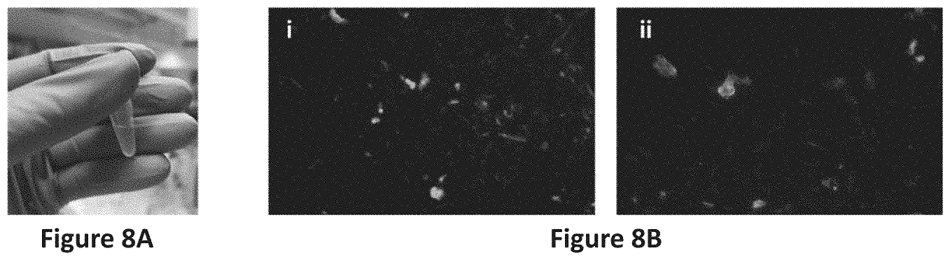

In some embodiments, another exemplary approach for 3D printing/bioprinting mediated by US polymerization (for all of the applications mentioned above) relies on crosslinker encapsulation and mixture preparation with pre-polymer solution. In some embodiments, the mixture is exposed to FUS which locally release the crosslinker, which induces local polymerization. In some embodiments, as described before, the FUS focal point is positioned spatially and print the designed object. In some embodiments, one example of this concept is the encapsulation of calcium in liposomes, which is then mixed with alginate for preparing acoustic-sensitive material, as shown for example in FIG. 8A and FIG. 8B (i-ii). In some embodiments, by applying FUS, the liposomes permeability increases, and the calcium is released locally and induce the ionic crosslinking of the alginate as part of patterned 3D printed alginate.

### Exemplary Materials and Methods

Acoustic-sensitive materials preparation: In some embodiments, for cavitation-based US polymerization synthetic materials (e.g. PEG-DA, HEMA, HAMA, PLA, PLGA, PCL, with functional acrylate or diacrylate or methacrylate groups) and/or natural materials (e.g. GelMA, collMa, alginate-MA, albumin-MA, chitosan-MA) with acrylate/methacrylate groups were used. For example, a 10% or 20% of PEG-DA dissolved in PBS or Water mixed for about 16 hr. In some embodiments, to achieve more biocompatible pH range between 7.2-7.5, PBS was used as buffer or adjusted with NaOH (1M). In some embodiments, this pH range also supports rapid and more efficient US mediated polymerization as a result of higher OH radical concentration as compared to lower pH of PEG-DA dissolved in double distilled water (ddW). In some embodiments, to increase biocompatibility for cell delivery application, one or more of the following composites were prepared: PEG-fibrinogen (1 mg/ml), PEG-DA composites: GelMA (5% or 10%), collagen (1 mg/ml), fibronectin (0.1 mg/ml), hydroxyapatite (0.5%, 1%, 2%). In some embodiments, for the preparation of viscous acoustic-sensitive materials for 3D printing application (by US or FUS induction), one or more of the following PEG-DA composites were prepared: alginate (0.1-2%), glycerol (20-70%), Pluronic F127 (5-30%).
US induction assay for polymerization: In some embodiments, for inducing polymerization by US application, a commercial (FDA approved) therapeutic US system with 1 MHz planar or focused transducers (0.3-2.2 watt power range) or 37 kHz sonication system was used. In some embodiments, the acoustic-sensitive materials pre-prepared with or without Ar saturation were exposed to US induction for the solvent polymerization. In some embodiments, full polymerization was achieved by an US exposure to a period of time of about 5 sec or longer.
US induced hydrogels characterization: In some embodiments, post US induced polymerization, the formed hydrogels were characterized for conversion rate by measuring the percentage of the unpolymerized solution post US induction, and the young modulus were calculated by analyzing the recorded force and strain measurements during 50% compression test via rheometer instrument. In some embodiments, in addition, hydrogels microstructures were captured using high magnification scanning electron microscope for freeze dried hydrogels samples pre-coated with gold nano-layer.
US induced polymerization for cell delivery assay: In some embodiments, for testing the potential cell delivery application using acoustic-sensitive materials platform, human dental pulp cells (DPSCs) were concentrated and mixed with the acoustic-sensitive solution (e.g. PEG-DA or the other biocompatible materials listed above) before rapid US induction using therapeutic US system (1 MHz, power 0.3-2.2 Watt/cm\hat{ }2, induction period 5-30 sec). In some embodiments, post polymerization the cell seeded hydrogels were cultivated within DPSCs growth medium in cell culture incubator (37° C., 5% CO₂).
Drug release assay: In some embodiments, for the drug release assay, a 1% (w/v) bovine serum albumin (BSA) was added to a PEG-DA pre-mixture. In some embodiments, a measured volume of this mixture was exposed to US for 20 seconds or 30 seconds to induce polymerization. In some embodiments, the resulting hydrogels were then incubated with fresh PBS in a humidified incubator (37° C., 5% CO₂). In some embodiments, the PBS was replaced after 1, 2, 4, 24, 48, 96, and 144 hours, and a sample was preserved for BSA concentration testing. In some embodiments, the BSA concentration in collected samples was measured using Coomasie blue Bradford assay kit (BioRad5000201) according to the manufacturers' instructions. In some embodiments, the cumulative sum of released BSA during the incubation period was calculated as shown in graph in FIG. 5C.
US mediated polymerization for 3D printing: In some embodiments, for 3D printing application viscous acoustic-sensitive material was prepared composed of 10% PEG-DA dissolved in 70% glycerol solution. In some embodiments, for 3D printing within supportive sacrificial substrate the acoustic-sensitive material was loaded into the printer syringe and the CAD models were printed layer by layer into Agar support bath via 300 um nozzle. In some embodiments, immediate after, the printed object was polymerized by US induction and the support material was removed. In some embodiments, to demonstrate the ability to print spatially directly by FUS a conic tube with a convex bottom was used, which concentrated the US waves into a spatial focal point from where it initiated a localized polymerization of the acoustic-sensitive viscous material. In some embodiments, another option to apply FUS spatially is to use convex US lens attached to the transducer or to mount guiding cone to concentrate the US waves into one focal point.
Calcium loaded liposomes preparation: In some embodiments, calcium-loaded liposomes were fabricated using an established known method. Briefly, a solution of DPPC (Avanti 850355P) was prepared in chloroform, dried with a stream of nitrogen gas in a glass vial and then kept under vacuum for at least 2 h. The lipid film was hydrated to a lipid concentration of 2 mg/ml with an aqueous CaCl2 solution for 1 h. Next, the mixture was vortexed and sonicated to produce unilamellar vesicles. For liposomes fluorescent visualization, fluorescein was added to the CaCl2 mixture. The vesicles were then dialyzed with isotonic solution without CaCl2. The calcium containing liposomes were added to alginate solution and mixed thoroughly prior to US induction.

### Exemplary General Method

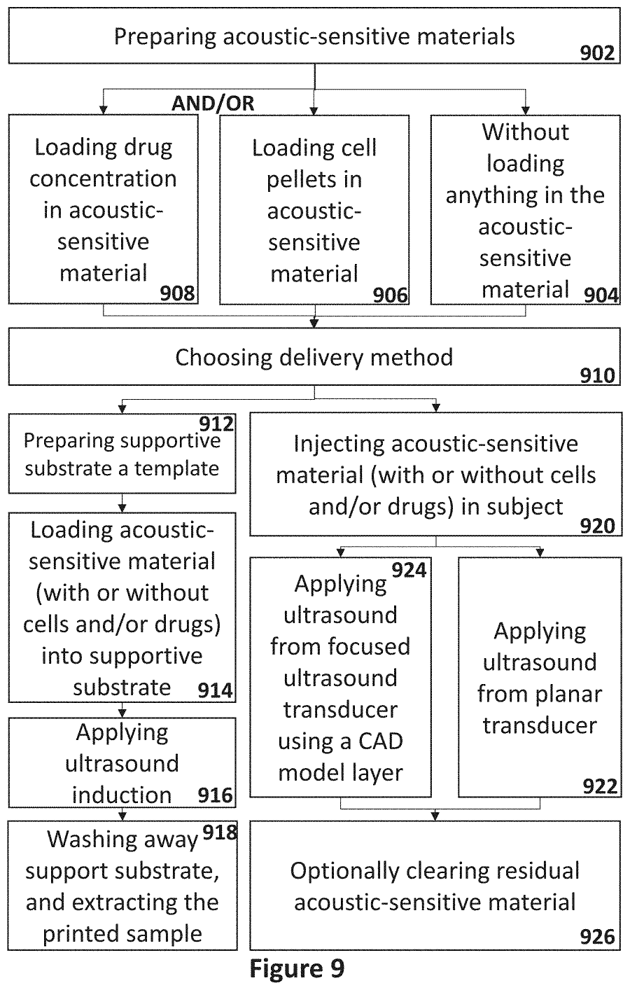

Referring now to FIG. 9, showing flowchart of exemplary methods, according to some embodiments of the invention. In some embodiments, ultrasound-mediated polymerization, optionally for cell and/or drug delivery procedure comprises the following actions:

Preparing of the acoustic-sensitive materials 902: In some embodiments, these materials comprise one or more and not limited to PEG-DA, PBS, Matrigel, PEG-fibrinogen, Hydroxyapatite, alginate, glycerol (in some embodiments can include other materials with functional diacrylate or methacrylate groups).

In some embodiments, acoustic-sensitive materials are used as is 904.

In some embodiments, for cell delivery application, cell pellets are added to the acoustic-sensitive mixture briefly before the application of ultrasound 906.

In some embodiments, for drug delivery applications, the chosen drug concentration is loaded into an acoustic-sensitive mixture before the application of ultrasound 908.

In some embodiments, one or both of cell types and drugs are added to the acoustic-sensitive mixture before the application of ultrasound.

In some embodiments, a delivery method is then chosen 910.

In some embodiments, for ultrasound printing within supportive substrate, a self-healing 3D supportive substrate is prepared in advanced using one or more of agar, gelatin, Pluronic F-127, or other support media 912, followed by 3D printing of the acoustic-sensitive mixture in the desired template 914, and followed by ultrasound application for polymerization 916. In some embodiments, applying ultrasound induction using low frequency transducers (from about 30 kHz to about 1000 kHz) for from about 5 seconds to about 30 seconds in order to reach polymerization with an intensity range of from about 0.5 Watt/cm to about 2.2 Watt/cm. In some embodiments, right after the ultrasound application, the support material is washed away, and the printed sample is extracted 918.

In some embodiments, for in-situ noninvasive printing the acoustic-sensitive mixture is injected into the area of interest 920, or injected through the veins of the patient, followed by either applying ultrasound from a planar transducer, thereby polymerizing without a specific pattern 922, or applying ultrasound with local patterning by applying focused ultrasound induction according to the CAD model layer by layer until polymerizing the full model shape 924. In some embodiments, applying ultrasound induction using low frequency transducers (from about 30 kHz to about 1000 kHz) for from about 5 seconds to about 30 seconds in order to reach polymerization with an intensity range of from about 0.5 Watt/cm to about 2.2 Watt/cm. In some embodiments, optionally, the residual unpolymerized material is cleared from the treated area using a minimally invasive collecting needle 926.

### Exemplary PVA-MA as Another Acousto-Sensitive Material

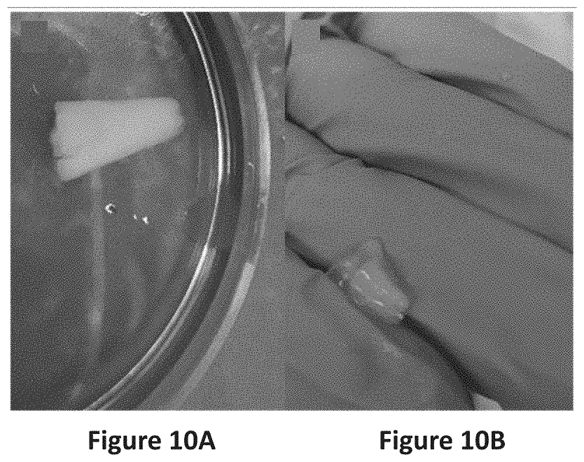

Referring now to FIGS. 10A-B showing exemplary PVA-MA material used, according to some embodiments of the invention. In some embodiments, a variation of the basic protocol uses polyvinyl alcohol PVA-MA solution instead of PEG-DA. In some embodiments, the PVA-MA is synthesized using PVA 30-70 kDa, 87-90% hydrolyzed. In some embodiments, the precipitation is performed in acetone. In some embodiments, after synthesis, a 10% PVA-MA solution is prepared, dissolving the dry PVA-MA in PBS, and the basic polymerization protocol is applied. In some embodiments, as shown for example in FIGS. 10A and 10B, the PVA-MA solution is successfully polymerized using US.

List of abbreviations

| Abbreviations | Full form |
|---|---|
| Albumin-MA | Albumin methacrylate |
| alginate-MA | alginate methacrylate |
| BSA | Bovine serum albumin |
| CAD | Computer-aided design |
| Chitosan-MA | Chitosan methacrylate |
| coll-MA | Collagen methacrylate |
| DPSC | Pental pulp stem cell |
| ECM | Extracellular matrix |
| FUS | Focused ultrasound |
| HAMA | Methacrylated Hyaluronic Acid |
| HEMA | (Hydroxyethyl)methacrylate |
| PBS | Phosphate buffered saline |
| PCL | Polycaprolactone |
| PEG-DA | Poly(ethylene glycol) diacrylate |
| PEG-Fibrinogen | Poly(ethylene glycol) - Fibrinogen |
| PLA | Polylactic acid |
| PLGA | Poly(lactic-co-glycolic acid) |
| PVA-MA | Polyvinyl alcohol methacrylate |
| SEM | Scanning electron microscope |
| US | Ultrasound |

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An implant, comprising:
a. acoustic-sensitive material, and
b. at least one additional component within said acoustic-sensitive material.

2. The implant according to claim 1, wherein said at least one additional component is one or more of at least one releasable drug within said acoustic-sensitive material and a plurality of cells within said acoustic-sensitive material.

3. The implant according to claim 1, wherein said acoustic-sensitive material comprises one or more of materials with functional acrylate or diacrylate or methacrylate groups, PEG-DA, polyvinyl alcohol PVA-MA, PBS, Matrigel, PEG-fibrinogen, Collagen, Fibronectin, Hydroxyapatite, alginate, glycerol.

4. The implant according to claim 1, wherein said acoustic-sensitive material hardens when exposed to ultrasound emissions.

5. The implant according to claim 4, wherein said ultrasound emissions are characterized by at least one selected from the group consisting of:
a. low frequencies;
b. frequencies from about 30 kHz to about 1000 kHz;
c. being emitted for a period of time of from about 3 seconds to about 120 seconds;
d. by an intensity range of from about 0.1 Watt/cm² to about 10 Watt/cm²;
e. any combination thereof.

6. The implant according to claim 1, wherein said implant is printed within a supportive subtract.

7. The implant according to claim 6, wherein said printed within said supportive material is performed before implantation of said implant or after implantation of said implant.

8. The implant according to claim 6, wherein said supportive material is characterized by one or more of:
a. comprising one or more of agar, gelatin and Pluronic F-127; and
b being washable away.

9. The implant according to claim 1, wherein said implant comprises a dedicated form when focused ultrasound is applied to said implant according to a predetermined CAD model layer.

10. The implant according to claim 1, wherein said acoustic-sensitive material comprises a solution of pre-polymer and acoustic-sensitive cross-linker loaded micro-capsules.

11. The implant according to claim 10, wherein said acoustic-sensitive cross-linker loaded micro-capsules comprise liposomes including said cross-linker.

12. The implant according to claim 10, wherein said pre-polymer comprises alginate.

13. An implant system, comprising:
a. an ultrasound transducer; and
b. an implant comprising:
i. acoustic-sensitive material, and
ii. at least one component within said acoustic-sensitive material.

14. The system according to claim 13, wherein said at least one component is one or more of at least one releasable drug within said acoustic-sensitive material and a plurality of cells within said acoustic-sensitive material.

15. The system according to claim 13, wherein said acoustic-sensitive material comprises one or more of materials with functional acrylate or diacrylate or methacrylate groups, PEG-DA, PVA-MA, PBS, HAMA, PCL, PLA, PLGA, Matrigel, PEG-fibrinogen, Collagen, Fibronectin, Hydroxyapatite, alginate, glycerol.

16. The system according to claim 13, wherein said acoustic-sensitive material hardens when exposed to ultrasound emissions provided by said ultrasound transducer.

17. The system according to claim 16, wherein said ultrasound emissions are characterized by at least one selected from the group consisting of:
a. low frequencies
b. frequencies from about 30 kHz to about 1000 kHz;
c. being emitted for a period of time of from about 3 seconds to about 120 seconds;
d. by an intensity range of from about 0.1 Watt/cm² to about 10 Watt/cm²;
e. any combination thereof.

18. The system according to claim 13, wherein said implant is printed within a supportive subtract.

19. The system according to claim 18, wherein said printed within said supportive material is performed before implantation of said implant or after implantation of said implant.

20. The system according to claim 18, wherein said supportive material is characterized by one or more of:
a. comprising one or more of agar, gelatin and Pluronic F-127;
b. being washable away.

21. The system according to claim 13, wherein said implant comprises a dedicated form when focused ultrasound is applied to said implant according to a predetermined CAD model layer.

22. The system according to claim 13, wherein said acoustic-sensitive material comprises a solution of pre-polymer and acoustic-sensitive cross-linker loaded micro-capsules.

23. The system according to claim 22, wherein said acoustic-sensitive cross-linker loaded micro-capsules comprise liposomes including said cross-linker.

24. The system according to claim 22, wherein said pre-polymer comprises alginate.

25. A method of implanting an implant on a patient, comprising:
a. implanting acoustic-sensitive material in a first site of said patient;
b. selectively hardening said acoustic-sensitive material by emitting acoustic energy to a second site of said patient.

26. The method according to claim 25, wherein said first site and said second site are the same site.

27. The method according to claim 25, wherein said first site and said second site are different sites.

28. The method according to claim 25, wherein said first site is one or more of an implantation target site and a blood vessel.

29. The method according to claim 28, wherein said second site is said implantation target site.

30. The method according to claim 25, wherein said acoustic-sensitive material comprises one or more of:
a. materials with functional acrylate or diacrylate or methacrylate groups;
b. PEG-DA, PVA-MA, PBS, HAMA, PCL, PLA, PLGA, PBS, Matrigel, PEG-fibrinogen, Collagen, Fibronectin, Hydroxyapatite, alginate, glycerol;
c. a plurality of cells within said acoustic-sensitive material; and
d. at least one releasable drug within said acoustic-sensitive material.

31. The method according to claim 25, wherein said emitting acoustic energy comprises one or more of:
a. emitting ultrasound emissions;
b. emitting at low frequencies;
c. emitting at a frequency of from about 30 kHz to about 1000 kHz;
d. emitting for a period of time of from about 3 seconds to about 120 seconds; and
e. emitting ultrasound emissions that are characterized by an intensity range of from about 0.1 Watt/cm² to about 10 Watt/cm².

32. The method according to claim 25, wherein said selectively hardening is performed within a supportive material.

33. The method according to claim 32, wherein said selectively hardening within said supportive material is performed before implantation of said implant of after implantation of said implant.

34. The method according to claim 32, wherein said method further comprises washing away said supportive material.

35. The method according to claim 25, wherein said method further comprises providing a dedicated form to said implant by emitting focused ultrasound to said implant according to a predetermined CAD model layer.

36. A method of generating an acoustic-sensitive implant comprising at least one cell, comprising:
a. adding said at least one cell into a hydrogel solution thereby generating a cell/hydrogel solution;
b. contemporarily injecting said cell/hydrogel solution and at least one oil via a dedicated syringe, thereby generating individual cell/hydrogel beads;
c. dropping said individual cell/hydrogel beads in a calcium chloride solution;
d. separating said individual cell/hydrogel beads from said calcium chloride solution;
e. adding said separated individual cell/hydrogel beads into a PEG-DA solution.
